(19) United States
(12) Patent Application Publication
(10) Pub. No.: US 2012/0107920 A1
(43) Pub. Date: May 3, 2012

(54) METHOD OF PRODUCING SUGAR SOLUTION AND SACCHARIFICATION DEVICE

(75) Inventors: Daisuke Taneda, Ibaraki (JP); Yoshiki Ueno, Ibaraki (JP); Hirokazu Konishi, Ibaraki (JP); Makoto Ikeo, Ibaraki (JP)

(73) Assignee: JGC CORPORATION, Chiyoda-ku (JP)

(21) Appl. No.: 13/261,131

(22) PCT Filed: Jul. 15, 2010
(86) PCT No.: PCT/JP2010/004608

§ 371 Date: Jan. 9, 2012

(30) Foreign Application Priority Data
Jul. 17, 2009 (JP) 2009-169527

(51) Int. Cl. C13B 50/00; C12M 1/00

(52) U.S. Cl. 435/276; 435/289.1

(57) ABSTRACT

To achieve efficient use of enzyme in obtaining a sugar solution through a reaction of the enzyme and biomass containing cellulose. In obtaining a sugar solution through a reaction of enzyme and biomass containing cellulose, the biomass and the enzyme are caused to react in a first reaction tank, whereby a sugar solution in which the enzyme is dispersed and a residue containing the unreached biomass adsorbing the enzyme are generated, these sugar solution and residue are next separated, a pH adjusting solution is supplied to the residue in a second reaction tank to prepare a dilute solution whose sugar concentration is lower than that of the sugar solution, and in this dilute solution, a sugar solution is generated through a reaction between the residue and the enzyme adsorbed to the residue.

### TECHNICAL FIELD

The present invention relates to a sugar solution producing method and a saccharification device for producing a sugar solution through a reaction between enzyme and biomass containing cellulose.

### BACKGROUND ART

In recent years, in order to obtain a substitution energy source for oil resources, there has been considered a method of producing alcohol by producing a sugar solution from biomass such as a woody or herbaceous material, for example, wood chips, rice straws, and so on with the use of enzyme and fermenting sugar of the sugar solution, as shown in Patent Document 1, for instance. Main components of the biomass of these kinds are: lignin which is an aromatic compound and has a three-dimensional structure; and cellulose and hemicellulose each being a sugar polymer. The cellulose and hemicellulose are hydrolyzed by the enzyme into a monosaccharide that will be a raw material for alcohol fermentation. However, the cellulose and the hemicellulose are protected by the lignin, and therefore, as shown in FIG. 25, after pretreatment, for example, dilute sulfuric acid decomposition or the like for breaking the lignin is performed, the enzyme such as cellulase is added to cause a reaction for several days, for instance, whereby a sugar solution of, for example, glucose being the raw material for the fermentation is obtained, and subsequently, fermentative microorganism is added to this sugar solution to cause its fermentation, whereby alcohol, for example, ethanol, being an end product is obtained. This alcohol is thereafter condensed (distilled) to a predetermined concentration as required, for instance.

As a concrete device for performing this process, used is a device, as shown in FIG. 26(a), for instance, which includes a saccharification tank 101 for producing a sugar solution and a fermentation reaction tank 102 for fermentation, and in which enzyme and biomass having undergone the pretreatment are put in the saccharification tank 101 to react with each other, next, the sugar solution obtained in the saccharification tank 101 is supplied together with a fermentative microorganism to the fermentation reaction tank 102 to be fermented into ethanol. The enzyme hydrolyzes cellulose and hemicellulose in the saccharification tank 101 to produce the sugar solution including a monosaccharide such as glucose or xylose.

In order to perform such a process as actual business on a payable basis, a great cost reduction is required, which involves the following problems. Firstly, for example, because the enzyme used to produce the sugar solution is very expensive, its efficient use by the recovery and reuse is necessary without discarding the enzyme every time the reaction is finished. For example, the above-described device is capable of collect the enzyme by, for example, membrane separation after the enzyme causes the reaction of the biomass in the saccharification tank 101, but in this case, the lifetime of the membrane becomes short because the membrane becomes dirty, which leads to a cost increase. Further, by the membrane separation, the enzyme adsorbed to a cracked residue of the biomass cannot be recovered. Therefore, a study to find a method for low-cost reuse of the enzyme as well as consumables such as this membrane is needed.

Further, in order to reduce an amount of the unreacted biomass discarded as the residue to reduce the waste of the raw material, it is necessary to make a ratio of the sugar solution produced from the supplied biomass (saccharification ratio: weight of generated sugar/weight of cellulose and hemicellulose in biomass) as high as possible. At this time, in the aforesaid enzymatic reaction, the presence of the sugar inhibits an enzymatic decomposition reaction, which gives rise to a problem that a reaction rate decreases as a sugar concentration increases in accordance with the progress of the reaction. Further, in the biomass, a portion that is easily enzymatically decomposed, such as, for example, an amorphous portion in the cellulose and a portion that is not easily decomposed such as, for example, a portion having high crystallinity in the cellulose are both present. In the enzymatic reaction of the biomass, it is thought that the decomposition first progresses in its easily decomposed portion, and it is thought that as the reaction progresses, a reaction rate further decreases because an amount of the easily decomposed portion reduces.

Therefore, if under a condition where the sugar concentration is high, an effort is made to make the cellulose and the hemicellulose react completely or almost completely for the purpose of increasing the saccharification ratio, the reaction takes many days and the running time of the device increases contrary to the intension, leading to a cost increase. Further, an effort to increase the saccharification ratio by supplying a large amount of the enzyme leads to a cost increase unless the enzyme is recovered for reuse.

Another possible method to increase the saccharification ratio is, for example, to reduce the sugar concentration by reducing a charge amount of the biomass into the reaction tank 101, thereby reducing an amount of the residue without requiring many days for the reaction, but in this case, since the concentration of the obtained sugar solution becomes low, the concentration of the fermentation ethanol also decreases. Accordingly, an energy amount required for the distillation at the time of the production of the condensed ethanol being an end product increases and an installed capacity becomes large. Furthermore, in order to produce the sugar solution low in sugar concentration, the saccharification tank 101 and the enzymatic reaction tank 102 need to be large, which leads to a cost increase. Therefore, it is very difficult to increase the saccharification ratio at low cost without spending many days for the reaction.

Under such circumstances, there has been studied a method in which the enzymatic reaction and the ethanol fermentation both take place in the same reaction tank 103 as shown in FIG. 26(b). This method is a method in which biomass and enzyme are supplied into the reaction tank 103, and next, a fermentative microorganism is supplied to the reaction tank 103, so that a sugar solution obtained through the reaction between the biomass and the enzyme is fermented by the fermentative microorganism as necessary to be turned into ethanol. In this method, the concentration of the sugar solution being a factor inhibiting the saccharification is held down, so that the sugar inhibition becomes small, which is thought to be a reason why the saccharification ratio can be increased. However, an optimum reaction temperature of the enzymatic reaction is generally 50° C. to 60° C., while an optimum reaction temperature of the fermentation is 30° C. to 35° C. because of heat tolerance of the fermentative microorganism. Therefore, in this method, the reaction needs to take place at 30° C. to 35° C. in order to prevent the extinction of the fermentative microorganism, which lowers reactivity of the enzyme. Accordingly, an amount of the supplied enzyme increases or the reaction takes a long time on the contrary.

Further, in order to obtain the ethanol from the biomass at low cost by the above-described enzymatic method, it is desirable to produce the sugar solution being a precursor of the ethanol so that it has as high concentration as possible, for the purpose of, for instance, reducing energy required for condensing the ethanol, but it is extremely difficult to obtain the high-concentration sugar solution due to the sugar inhibition, or because the biomass low in reactivity remains in a terminal period of the reaction as described above. It is known that a high-concentration sugar solution can be obtained by, for example, increasing a supply amount of the enzyme and a charge amount of the biomass in the reaction tank 101, but in this method, an amount of the used enzyme increases and further an amount of the unreacted residue increases, leading to a high cost.

Patent Document 2 describes an art in which, in concurrent saccharification-fermentation reactions where cellulose is turned into ethanol, a liquid part including enzyme and alcohol is brought into contact with a solid including the cellulose contributing to the concurrent saccharification-enzymatic reactions, whereby the enzyme is adsorbed to the solid, but this method does not consider the production of a high-concentration sugar solution.

### Patent Document 1

Japanese Patent Application Laid-open No. 2006-87319

### Patent Document 2

Japanese Patent Application Laid-open No. Sho 55-144885 (Claims and so on)

### DISCLOSURE OF THE INVENTION

### Problems to Be Solved by the Invention

The present invention was made under such circumstances, and has an object to provide a sugar solution producing method and a saccharification device that make it possible to obtain a high-concentration sugar solution at low cost when obtaining a sugar solution through a reaction between enzyme and biomass including cellulose.

### Means for Solving the Problems

A method of producing a sugar solution of the present invention is a method to obtain a sugar solution through a reaction of cellulose-based biomass being an aggregate of plants or an aggregate of processed products of plant including cellulose with a saccharifying enzyme having cellulose saccharifying ability, the method including:

a first reaction step of mixing a saccharifying enzyme and cellulose-based biomass in an aqueous solution to cause a saccharification of the biomass by the saccharifying enzyme;

a first separation step of causing solid-liquid separation of a reaction solution obtained in the first reaction step to obtain a sugar solution and a residue; and

a second reaction step of preparing an aqueous solution by adding additive water to the residue obtained in the first separation step, and in the aqueous solution, causing a saccharification of the residue by the saccharifying enzyme adsorbed to the residue; and

a third reaction step of adding cellulose-based biomass not having undergone the saccharification to the sugar solution obtained in the first separation step and causing a saccharification of the newly added biomass by the saccharifying enzyme present in the sugar solution.

Different reaction tanks are preferably used for the first reaction step, the second reaction step, and the third reaction step.

Preferably, the method includes:

a second separation step of causing solid-liquid separation of a reaction solution obtained in the second reaction step to obtain a sugar solution and a residue;

a third separation step of causing solid-liquid separation of a reaction solution obtained in the third reaction step to obtain a high-concentration sugar solution and a residue; and

a reaction step of mixing the sugar solution obtained in the second separation step and the residue obtained in the third separation step to cause a saccharification,

wherein the reaction step corresponds to the first reaction step, and the biomass used in the first reaction step is the residue obtained in the third separation step.

Preferably, the method further includes a step of additionally supplying a saccharifying enzyme to at least one of the aqueous solution in the second reaction step and the sugar solution separated in the second separation step.

Preferably, a ratio of lignin contained in the biomass is 10% or less.

A saccharification device of the present invention is a saccharification device for carrying out the above-described method of producing the sugar solution, the device including:

two reaction tanks in each of which a saccharifying enzyme and a cellulose-based biomass are mixed in an aqueous solution and a saccharification of the biomass is caused by the saccharifying enzyme;

a biomass supply part to supply the biomass into the reaction tanks;

a first enzyme supply part to supply the saccharifying enzyme into the reaction tanks;

an additive water supply part to supply additive water into one reaction tank out of the two reaction tanks;

a first separating part to perform solid-liquid separation of a reaction solution generated by the reaction between the biomass and the saccharifying enzyme in the one reaction tank to obtain the sugar solution and the residue;

a first transfer part to transfer the sugar solution separated by the first separating part to the other reaction tank out of the two reaction tanks; and

a control part that outputs a control signal so that the first reaction step is performed in the one reaction tank, the sugar solution obtained by the first separating part is next supplied to the other reaction tank, thereafter the additive water and cellulose-based biomass not having undergone the saccharification are supplied into the reaction tank storing the residue and the reaction tank storing the sugar solution respectively, and the second reaction step and the third reaction step are performed in the two reaction tanks respectively.

The first enzyme supply part may supply the saccharifying enzyme into the one reaction tank instead of the two reaction tanks,

the additive water supply part may supply the additive water into the other reaction tank instead of the one reaction tank,

the first transfer part may transfer the residue instead of the sugar solution to the other reaction tank, and

the control part may output the control signal so that after the first reaction step is performed, the other reaction tank is supplied with the residue obtained by the first separating part instead the sugar solution obtained by the first separating part.

The first separating part may separate the sugar solution and the residue by sedimentation in the one reaction tank.

A saccharification device of the present invention is a saccharification device for carrying out the method of producing the sugar solution described above, the device including:

a first reaction tank, a second reaction tank, and a third reaction tank in each of which a saccharifying enzyme and a cellulose-based biomass are mixed in an aqueous solution and a saccharification of the biomass is caused by the saccharifying enzyme;

biomass supply parts to supply the biomass to the first reaction tank and the third reaction tank respectively;

an enzyme supply part to supply the saccharifying enzyme into the first reaction tank;

an additive water supply part to supply additive water into the second reaction tank;

a first separating part to perform solid-liquid separation of a reaction solution obtained by the reaction between the saccharifying enzyme and the biomass in the first reaction tank to obtain a sugar solution and a residue;

a first sugar solution supply part and a first residue supply part to supply the sugar solution and the residue separated by the first separating part to the third reaction tank and the second reaction tank respectively; and

a control part that outputs a control signal so that the first reaction step is performed in the first reaction tank, next, the residue and the sugar solution are separated by the first separating part from the reaction solution obtained in the first reaction tank, the residue and the sugar solution are supplied to the second reaction tank and the third reaction tank respectively, and thereafter, the additive water and cellulose-based biomass not having undergone the saccharification are supplied to the second reaction tank and the third reaction tank respectively, and the second reaction step and the third reaction step are performed in the reaction tanks respectively.

Preferably, the device includes:

a second separating part to perform solid-liquid separation of a reaction solution generated in the second reaction tank to obtain a sugar solution and a residue;

a third separating part to perform solid-liquid separation of a reaction solution generated in the third reaction tank to obtain a high-concentration sugar solution and a residue;

a second sugar solution supply part to supply the first reaction tank with the sugar solution separated in the second separating part;

a third residue supply part to supply the first reaction tank with the residue separated in the third separating part; and

a sugar solution collecting part to take out the high-concentration sugar solution separated in the third separating part,

wherein there is performed a step of obtaining a sugar solution and a residue from a reaction solution obtained in the second reaction tank, by using the second separating part, obtaining a high-concentration sugar solution and a residue from a reaction solution obtained in the third reaction tank, by using the third separating part, and making the sugar solution separated in the second separating part react in the first reaction tank with the residue separated in the third separating part, and

wherein the step corresponds to the first reaction step, and the biomass used in the first reaction step is the residue separated in the second separating part.

The first separating part, the second separating part, and the third separating part may separate the sugar solution and the residue by sedimentation in the first reaction tank, the second reaction tank, and the third reaction tank respectively.

Preferably, the biomass is subjected to a pretreatment operation by an appropriate method, and a ratio of lignin contained in the biomass is 10% or less, or the biomass is a processed product of a plant material with a high cellulose content ratio and a low lignin content ratio, such as used paper, pulp, cotton, or cotton fiber.

### Effect of the Invention

In the present invention, in obtaining a high-concentration sugar solution through a reaction between enzyme and biomass including cellulose, a sugar solution (I) and a residue (I) including unreacted biomass by which the enzyme is adsorbed are generated through a reaction between the biomass and the enzyme, next, the sugar solution (I) and the residue (I) are separated, a pH adjusting solution is supplied to the residue (I), and through a reaction between the residue (I) and the enzyme adsorbed to the residue (I), a sugar solution (II) is generated. Further, new biomass is supplied to the sugar solution (I), and through a reaction between the biomass and the enzyme remaining in the sugar solution (I), a sugar solution (III) and a residue (III) are generated. Further, by making the residue (III) and the sugar solution (II) react with enzyme contained in these residue (III) and sugar solution (II), a monosaccharide is further produced. This enables the effective use of the enzyme to reduce an amount of the discarded residue, which makes it possible to obtain a high-concentration sugar solution at low cost.

### BRIEF DESCRIPTION OF DRAWINGS

FIG. 1 is a side view showing an example of a sugar solution producing device of the present invention.

FIG. 2 are schematic views showing an example of biomass used in the sugar solution producing device.

FIG. 3 is a flowchart showing the flow of steps in the sugar solution producing device.

FIG. 4 are schematic views showing an example of the operation in the sugar solution producing device.

FIG. 5 is a schematic view showing how the biomass is decomposed.

FIG. 6 are schematic views showing an example of the operation in the sugar solution producing device.

FIG. 7 are schematic views showing an example of the operation in the sugar solution producing device.

FIG. 8 are schematic views showing an example of the operation in the sugar solution producing device.

FIG. 9 are schematic views showing an example of the operation in the sugar solution producing device.

FIG. 10 is a side view showing another example of the sugar solution producing device.

FIG. 11 is a side view showing another example of the sugar solution producing device.

FIG. 12 are schematic views showing an example of the operation in the sugar solution producing device in the other example.

FIG. 13 are schematic views showing an example of the operation in the sugar solution producing device in the other example.

FIG. 14 is a characteristic chart obtained in an example of the present invention.

FIG. 15 are characteristic charts obtained in the example of the present invention.

FIG. 16 is a characteristic chart obtained in the example of the present invention.

FIG. 17 is a characteristic chart obtained in the example of the present invention.

FIG. 18 is a characteristic chart obtained in the example of the present invention.

FIG. 19 is a characteristic chart obtained in the example of the present invention.

FIG. 20 is a characteristic chart obtained in the example of the present invention.

FIG. 21 is a characteristic chart obtained in the example of the present invention.

FIG. 22 is a characteristic chart obtained in the example of the present invention.

FIG. 23 is a characteristic chart obtained in the example of the present invention.

FIG. 24 is a characteristic chart obtained in the example of the present invention.

FIG. 25 is a schematic chart showing an example of steps of obtaining ethanol from biomass.

FIG. 26 are side views of devices conventionally used to obtain ethanol from biomass.

### BEST MODE FOR CARRYING OUT THE INVENTION

(Device Structure of First Embodiment)

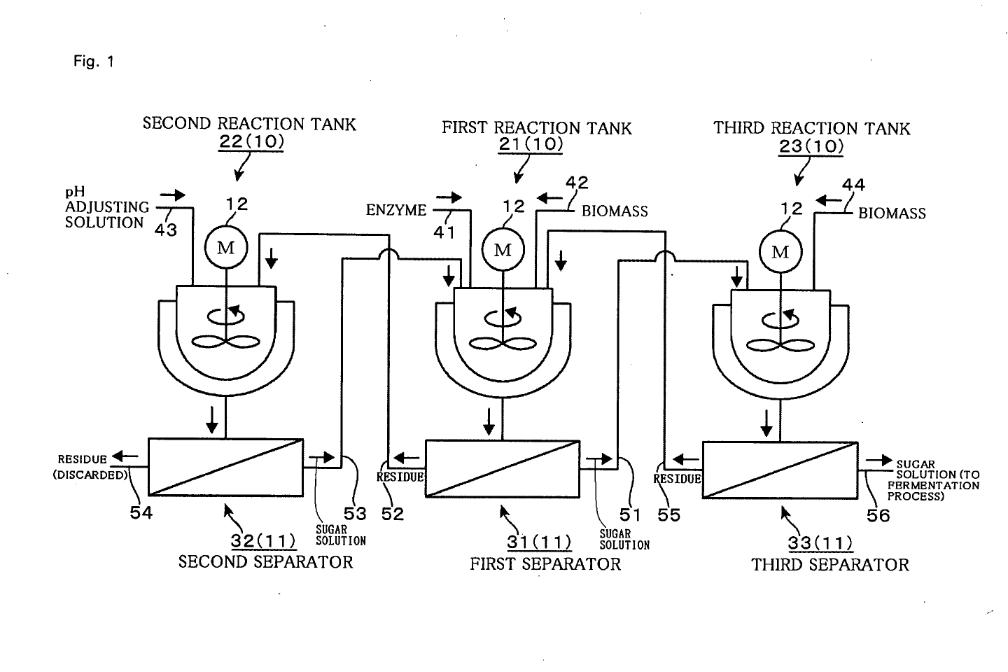

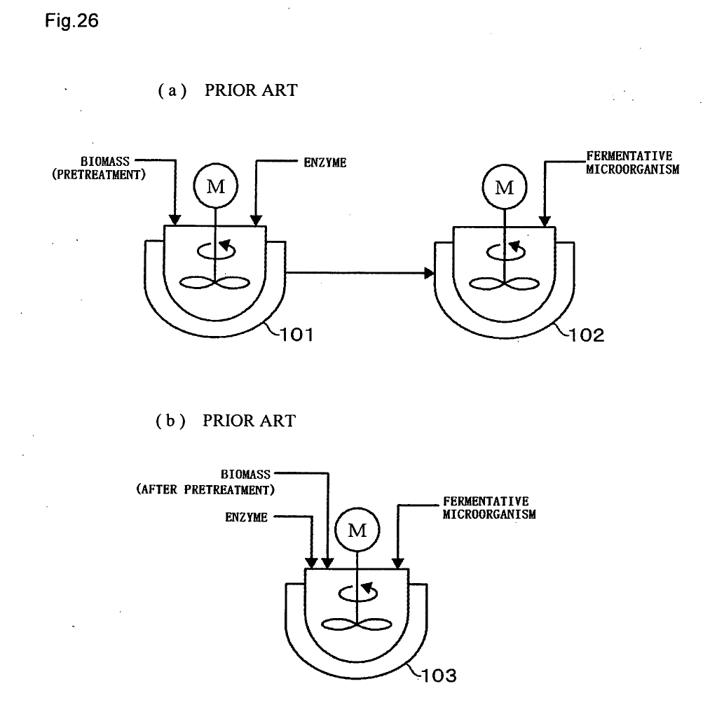

A first embodiment of a sugar solution producing device for performing a sugar solution producing method of the present invention will be described with reference to FIG. 1. The sugar solution producing device (saccharification device) is a device to produce a sugar solution (a solution in which a monosaccharide is dissolved) through a reaction of enzyme with biomass as a fermentation raw material including cellulose comprised of a polymer of glucose, and compared with, for example, the previously described device shown in FIG. 26, it is structured so that a concentration of sugar contained in the sugar solution taken to the outside becomes higher, an amount of a residue including the unreacted biomass discharged to the outside is smaller, that is, a saccharification ratio is higher, and an amount of the enzyme discharged to the outside together with the sugar solution and the residue is reduced. A reason why this device is structured as described above will be detailed later, and firstly, the structure of the device will be briefly described. This device includes: three reaction tanks 10; and separators 11 being separating parts which are provided for the respective reaction tanks 10 and each of which includes a device for separating the sugar solution and the residue containing the unreacted biomass which are generated in the corresponding reaction tank 10, for example, a sedimentation separator, a pressure filtration device, a suction filtration device, a centrifugal separator, a cyclone separator, or a filter press device.

The reaction tanks 10 are each provided with a heater for keeping a reaction solution therein at a reaction temperature of, for example, about 50° C. to 60° C., a thermocouple for measuring the temperature of the reaction solution, and so on (none is shown). Further, in the drawing, a screw-type agitator is shown, but this does not limit the type of the agitator. For convenience sake, the reaction tank 10 at the center, the reaction tank 10 on the left, and the reaction tank 10 on the right in FIG. 1 are called a first reaction tank 21, a second reaction tank 22, and a third reaction tank 23 respectively. Further, the separators 11 connected to the first reaction tank 21, the second reaction tank 22, and the third reaction tank 23 respectively are also called a first separator 31, a second separator 32, and a third separator 33 respectively. In FIG. 1, 12 denotes agitators each agitating the reaction solution containing enzyme and biomass in the reaction tank 10 by, for example, an impeller, an agitating pump, air agitation, or the like, and for example, their agitation powers, agitating speeds, and so on are independently adjusted according to a ratio of solid (biomass and a residue) and liquid (sugar solution and pH adjusting solution) supplied into each of the reaction tanks 10.

In FIGS. 1, 41, 42, 43, and 44 denote an enzyme supply path, a biomass supply path, a pH adjusting solution supply path, and a biomass adding path respectively, and these paths are structured to be capable of supplying the first reaction tank 21 with a liquid (aqueous solution) containing the enzyme such as, for example, cellulase, for example, water adjusted to about pH 5 (pH adjusting solution), and the biomass containing cellulose, and capable of supplying the second reaction tank 22 with water adjusted to, for example, about pH5, which is additive water, and capable of supplying the third reaction tank 23 with the biomass as is supplied to the first reaction tank 21. The above enzyme supply path 41, biomass supply path 42 (biomass adding path 44), and pH adjusting solution supply path 43 constitute an enzyme supply part, a biomass supply part, and an additive water supply part respectively. These supply paths 41 to 43 (adding path 44) are equipped with not-shown valves for allowing/stopping the supply of the enzyme, the biomass, and the pH adjusting solution. Further, the aforesaid additive water is to prepare a low sugar concentration solution as will be described later, and may be, for example, a buffer solution, pure water, or the like other than the pH adjusting solution. In this device, to supply (transfer) a solid such as the biomass, a screw feeder is used, for instance, and a belt conveyor or the like is sometimes used in combination with the screw feeder. This also applied to a later-described residue.

A first sugar solution supply path 51 and a first residue supply path 52 for supplying the sugar solution and the residue separated in the separator 31 to the third reaction tank 23 and the second reaction tank 22 respectively are connected as a first sugar solution supply part and a first residue supply part to the first separator 31. Further, a second sugar solution supply path 53 being a second sugar solution supply part for supplying the first reaction tank 21 with the sugar solution separated in the separator 32 and a residue discharge path 54 for discarding the residue separated in the second separator 32, to the outside of the system are connected to the second separator 32. The residue discharged from the residue discharge path 54 is lignin 3 and so on contained in the biomass, and is, for example, burned so that thermal energy is recovered. A third residue supply path 55 supplying the first reaction tank 21 with the residue separated in the separator 33 and a sugar solution recovery path 56 taking the sugar solution separated in the separator 33 to the outside are connected as a third residue supply part and a sugar solution collecting part respectively to the third separator 33, and the sugar solution recovered from the sugar solution recovery path 56 is used as a raw material of a chemical product, or is fermented by a fermentative microorganism in a not-shown fermentation tank to turn into alcohol, for example, an ethanol solution. Then, this ethanol solution is thereafter condensed (distilled) for refinery. The above separators 31 to 33 are equipped with not-shown valves, which can allow/stop the supply of the residue and the sugar solution discharged from these separators 31 to 33.

(Regarding Biomass and Enzyme)

The biomass supplied to the first reaction tank 21 and the third reaction tank 23 is woody or herbaceous biomass whose lignin is broken or melted and removed by appropriate pretreatment. A method of the pretreatment is, for example, dilute sulfuric acid decomposition, steam explosion, ammonia explosion, supercritical ammonia treatment, hydrothermal/supercritical water treatment, biodegradation, pulverization, or chemical treatment. A content of the lignin in the biomass is, for example, 10% or less, preferably 5% or less. Further, other than the biomass having undergone the pretreatment, plant processed products such as used paper, pulp, cotton, and cotton fiber also become the biomass to be processed.

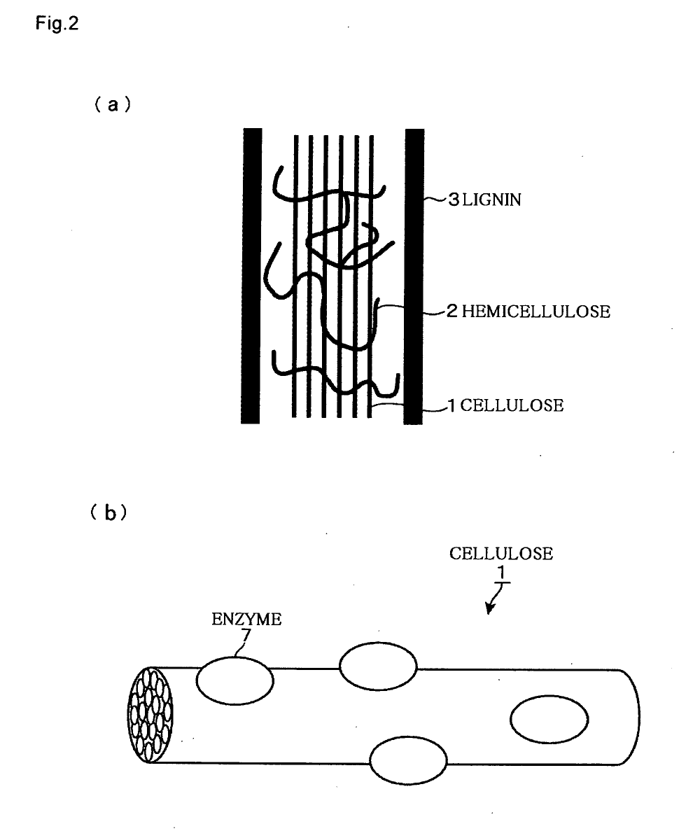

The enzyme supplied to the first reaction tank 21 is a cellulose decomposing enzyme or a hemicellulose decomposing enzyme, and is a particle (solid) with about several tens Å. Further, as shown in FIG. 2(b), these enzymes are adsorbed on surfaces of the cellulose 1 and the hemicellulose 2 to decompose the cellulose 1 and the hemicellulose 2 into monosaccharides, and the enzymes have a property to disperse in the reaction solution to flow like a liquid together with the reaction solution before adsorbed to the cellulose 1 and the hemicellulose 2 or after decomposing them. The enzyme 7 is a very expensive substance.

(Result of Experiment in Example and Study)

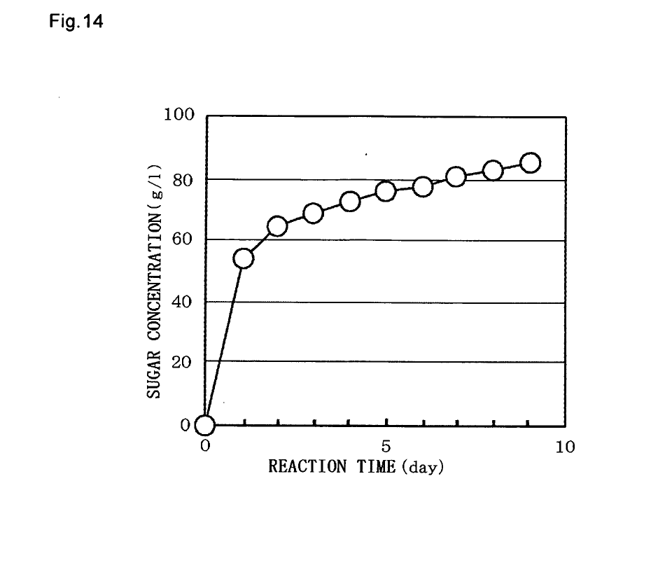

Next, a reason why the concentration of the sugar contained in the sugar solution taken to the outside is high, an amount of the residue discharged to the outside is small, and an amount of the enzyme discharged together with the sugar solution and the residue to the outside is reduced in the above-described device, compared with the aforesaid device shown in FIG. 26 will be described in detail based on experiment results shown in FIG. 14 to FIG. 24 in the following example and studies thereof. Regarding these FIG. 14 to FIG. 24, detailed experiment conditions and results will be described in the example to be described later.

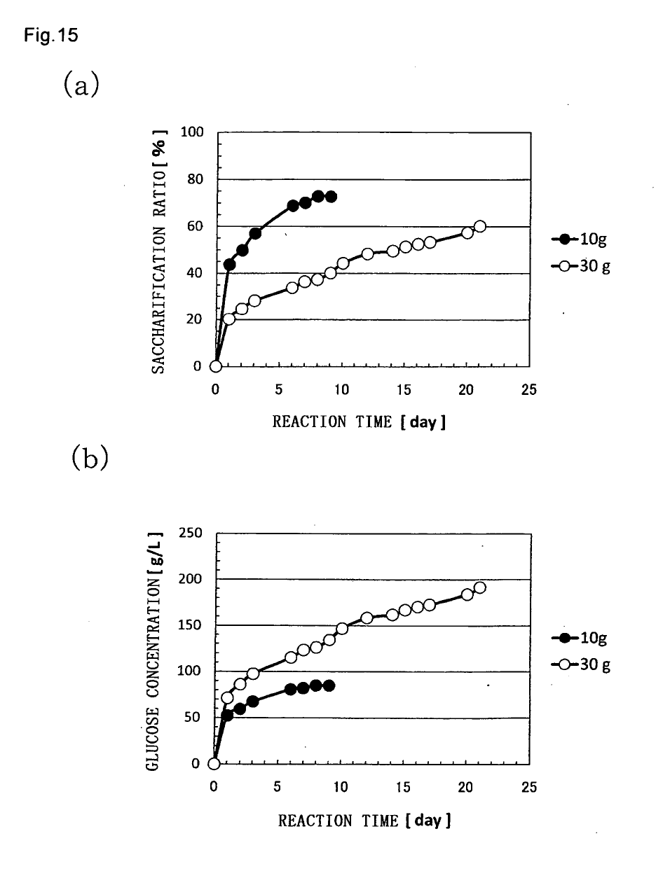

FIG. 14 is the result of an experiment conducted for confirming how the sugar concentration in a reaction solution increases when biomass and enzyme react with each other in the reaction solution (pH adjusting solution), and it is seen that an increasing rate of the sugar concentration (decomposition rate of the biomass) decreases with time. It is thought that a cause of such a decrease in the decomposition rate is that the presence of the sugar inhibits an enzymatic reaction, and the decomposition of the biomass starts from its easily decomposed portion, and therefore, in the reaction solution, the sugar concentration and a ratio of the biomass not easily decomposed increase with time (with the progress of the reaction). Here, weight of generated sugar/weight of the cellulose 1 and the hemicellulose 2 in the biomass is called a saccharification ratio, and as shown in FIG. 15(a), only an about 75% saccharification ratio (a supply amount of the biomass: 10 g) is conventionally obtained even when a long time was spent for the reaction as described above, and the remaining about 25% biomass is discarded as a residue, for instance. Further, increasing an amount of the charged biomass in order to increase the sugar concentration leads to an increase in the obtained sugar concentration as shown in FIG. 15(b), but leads to a further decrease in the saccharification ratio as shown in FIG. 15(a), so that an amount of the biomass wastefully discarded increases. From this, it is seen that the method in which an enzymatic reaction progresses in one reactor has a difficulty in producing a high-concentration sugar solution without any increase in an amount of a residue. Note that filter paper is used as the biomass in this experiment as will be described in the later-described example. This also applies to the following experiments. Therefore, it is thought that as the aforesaid residue, cellulose in the portion that does not easily react is left and lignin or the like that cannot be decomposed by the enzyme is not contained.

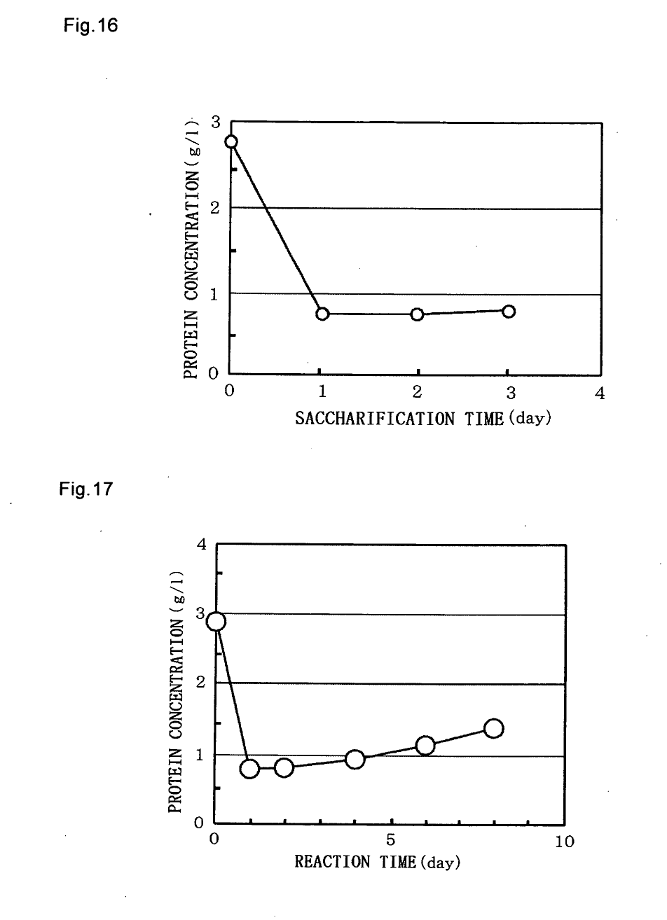

FIG. 16 is a result when measurement is conducted to see how enzyme concentration in a buffer solution changes with time when biomass is supplied into the buffer solution. From FIG. 16, when the biomass is supplied into the buffer solution, the enzyme concentration in the buffer solution decreases, which indicates that the enzyme in the buffer solution is adsorbed to the biomass. When this state is further confirmed for a longer time, it is seen that the enzyme once adsorbed to the biomass thereafter returns to the buffer solution again as time passes as shown in FIG. 17. That is, it is thought that the enzyme returns to the buffer solution when the enzyme cannot remain adsorbed to the biomass because the biomass decomposes to be saccharified (liquefied). From this, it is seen that it is possible to collect the enzyme present in a liquid such as the buffer solution by supplying the biomass to make the enzyme adsorbed to the biomass, and it is possible to collect the enzyme adsorbed to the biomass by decomposing the biomass. Note that in this experiment, protein in the buffer solution is regarded as the enzyme.

Further, from the above result in FIG. 17, it is seen that when the biomass decomposes due to the enzymatic reaction, the enzyme adsorbed to the biomass returns to the solution, but the whole quantity does not return. That is, it is seen that the enzyme is adsorbed to the unreacted biomass (residue) that does not easily decompose, in a reaction terminal period in FIG. 14. However, even when the enzyme is thus adsorbed, the decomposition of the biomass remaining as the residue does not quickly progress. In addition to the aforesaid reason that a ratio of the portion that does not easily decompose becomes large in the residue, a possible reason why the reaction of the residue does not quickly progress is that the sugar solution filled around the residue inhibits the enzymatic reaction and the decomposition reaction of the biomass such as the residue that does not easily decompose has a further difficulty in progressing. Therefore, it was studied for verification whether the reaction progressed when the residue was put in a solution where the influence of the sugar inhibition was small, that is, when the residue was separated from the sugar solution when the reaction did not easily progress because the enzymatic reaction had already progressed to a certain degree, and the residue was put into a state where the influence of the sugar inhibition was small (the separated residue was mixed with a buffer solution not containing sugar).

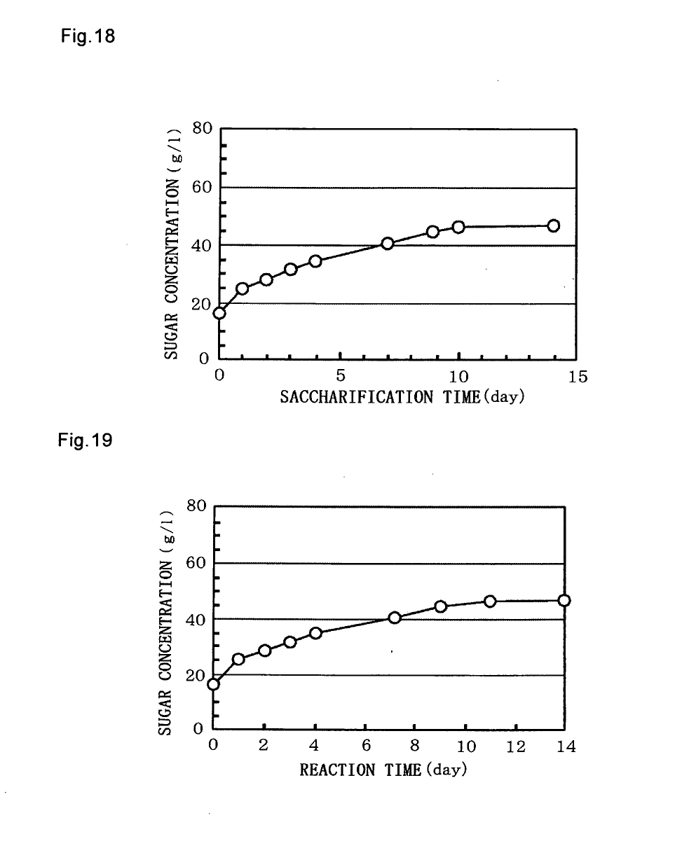

FIG. 18 and FIG. 19 are results obtained when a residue is separated from a sugar solution 168 hours later and 144 hours later from the start of the reaction, a buffer solution is added to the residue, and a sugar concentration in the solution is measured. From these results, it has been found out that even the residue whose reaction does not easily progress in the sugar solution before the separation quickly undergoes the reaction in the solution where the influence of the sugar inhibition is small (low sugar concentration solution). At this time, enzyme was not added to the buffer solution because it was thought from the aforesaid results in FIG. 16 and FIG. 17 that the enzyme had already been adsorbed to the residue, and even in this case, the enzymatic reaction progressed, which has led to the understanding that even the enzyme which does not easily cause the reaction due to the sugar inhibition can sufficiently contribute to the reaction in the low sugar concentration solution.

Therefore, when the foregoing results in FIG. 14 to FIG. 19 are put together, it is seen that an amount of the residue of the biomass reduces through so-called two-stage reaction processes where the unreacted biomass (residue) is separated from the sugar solution after the reaction of the enzyme and the biomass and the reaction of this biomass is caused in the solution whose sugar concentration is low. However, since the sugar concentration of the sugar solution obtained through the enzymatic decomposition of this residue is low, it is necessary to increase the sugar concentration of the sugar solution by making the sugar solution react with new biomass or residue (a residue containing a larger amount of easily decomposed portions than the aforesaid residue).

Next, as described above, the sugar solution obtained through the decomposition of the biomass is thereafter turned into ethanol by fermentation, and thereafter is condensed by, for example, distillation. Therefore, for the low-cost production of ethanol being an end product, a reduction in energy required for the condensation is required. Therefore, the concentration of the sugar contained in the sugar solution is desirably increased as much as possible. In order to increase the sugar concentration in the sugar solution without spending a long reaction time, an increase of an addition amount of the enzyme is necessary. However, increasing the addition amount of the enzyme results in higher production cost because in a conventional enzyme saccharification method, the expensive enzyme is discarded when it is once used. As a solution to this, there has been proposed an enzyme recovery method using a separation membrane, but this method needs a great facility expense for the separation membrane. Therefore, in the present invention, the following experiments and studies were conducted in order to configure a measure that can efficiently collect and reuse the added enzyme by a simple method even if an addition amount of the enzyme is increased in order to obtain a high-concentration sugar solution, can make this operation contribute to an increase in the concentration of the sugar solution, and can reduce an amount of the discarded residue.

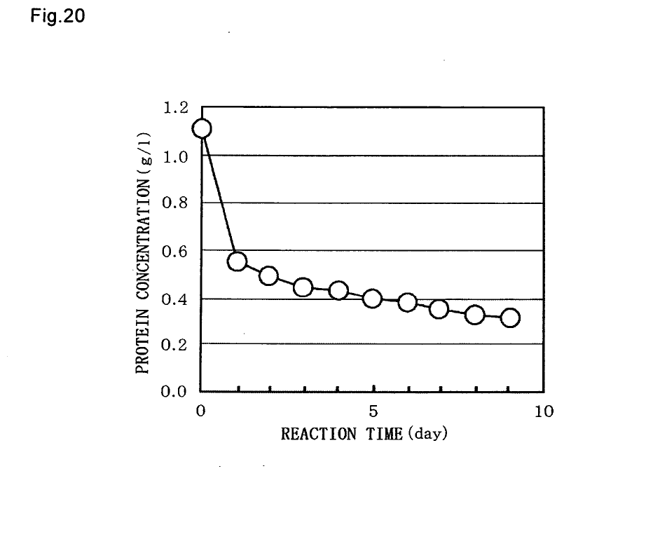

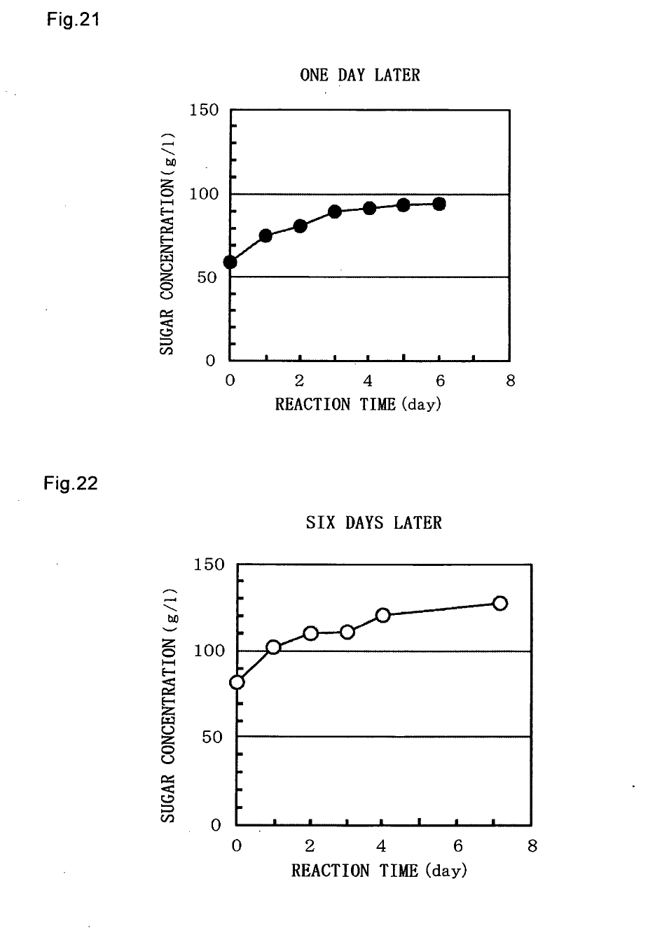

FIG. 20 is a result when a supernatant liquid (sugar solution) is centrifuged from the residue six days later from the start of the reaction in FIG. 14, biomass is added to the supernatant liquid, and a concentration of protein (enzyme) in the supernatant liquid is measured. From this result, it is seen that a large amount of the enzyme exists in the sugar solution before the start of the experiment, but after the biomass is added, an amount of the enzyme in the sugar solution decreases with time. That is, it can be said that the enzyme in the sugar solution is adsorbed to the biomass. Here, when the biomass was added to the supernatant liquid one day later and six days later from the start of the reaction and the sugar concentration was measured, the sugar concentration quickly increased in the both experiments as shown in FIG. 21 and FIG. 22. However, from the experiments so far, it is known that, even though the enzyme exists in this supernatant liquid, the enzymatic decomposition of the residue in which a ratio of portions having a crystal structure that is not easily decomposed is large does not easily progress, due to the occurrence of the sugar inhibition. Therefore, it is thought that cellulose decomposed in this sugar solution is an easily decomposed portion contained in the newly added biomass. Because this reaction quickly took place, it can be said that it is possible to obtain the high-concentration sugar solution without spending a long reaction time, through the aforesaid two-stage reaction processes, that is, by further adding new biomass to the sugar solution whose sugar concentration has become high to a certain degree after the biomass decomposition reaction takes place once and causing a decomposition reaction of an easily decomposed portion in the biomass.

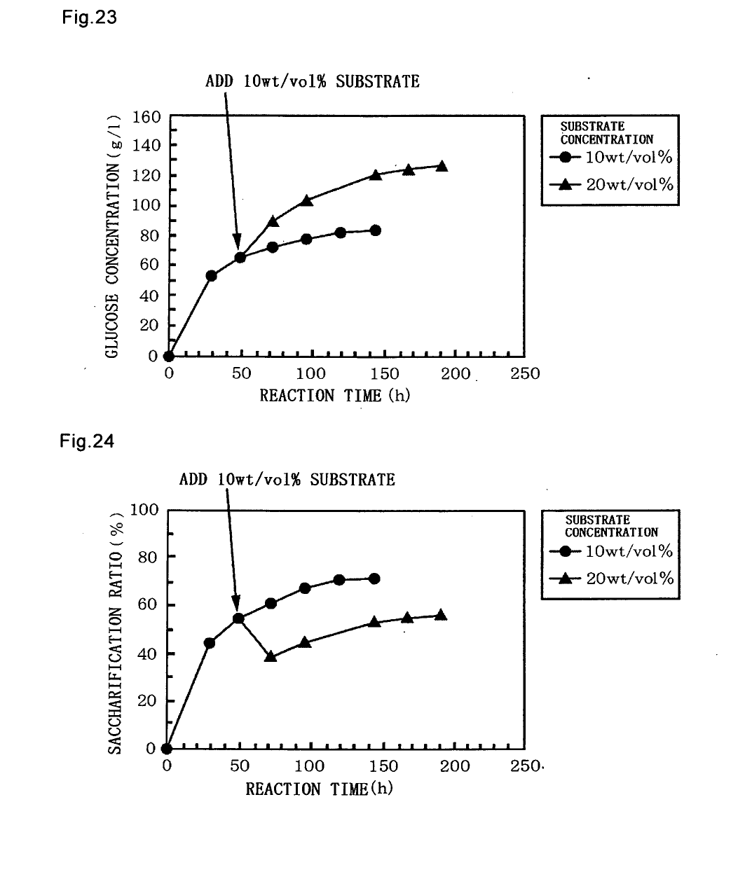

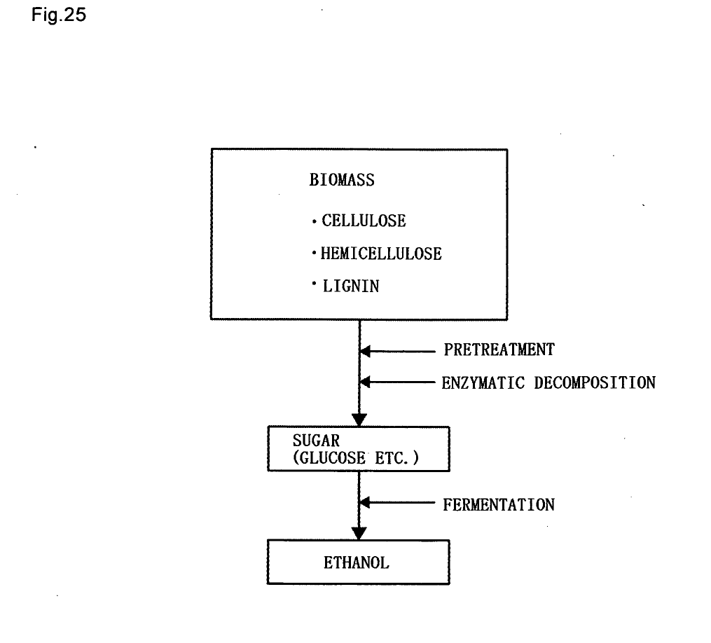

Here, when the new biomass is added, a method in which the residue and the sugar solution are not separated and the new biomass is added to them is also conceivable, instead of separating the residue and the sugar solution and adding the new biomass to this sugar solution. An experiment where new biomass is added 50 hours later from the start of the reaction without separating the sugar solution from the residue and the sugar concentration and the saccharification ratio are measured will be described with reference to FIG. 23 and FIG. 24. As shown in FIG. 23, the reaction rate (an increasing rate of the sugar concentration) is lower before the addition of the biomass than that at the initial period of the reaction, but after the biomass is added, the sugar concentration rapidly increases in a short time, and thus the same effects as those of the experiment results in FIG. 21 and FIG. 22 are confirmed even when the sugar solution and the residue are not separated. However, the saccharification ratio of the newly added biomass is lower than the initially supplied biomass because the newly added biomass is greatly influenced by the sugar inhibition, as is seen from FIG. 24. Therefore, it is seen that in order to obtain the high-concentration sugar solution at lower cost than the cost required by the aforesaid method shown in FIG. 15 (the method of increasing the saccharification ratio by increasing a charge amount of the biomass), it is necessary to also decompose the residue generated in this reaction. In this case, it can be thought that the method of supplying the residue into a low sugar concentration solution can be adopted as previously described.

However, when the biomass is newly added without separating the residue and the sugar solution as shown in FIG. 23 and FIG. 24, the residue with a high decomposition ratio (the residue of the biomass raw material first decomposed) and the residue with a low decomposition ratio (the residue of the biomass raw material added later and decomposed) mix with each other. Therefore, if an effort is made to cause the residue generated by the above reaction to react in the low sugar concentration solution, the residue generated from the newly added biomass first decomposes because this residue includes a portion that relatively easily reacts, and the decomposition of the residue generated from the initially supplied biomass does not progress much. That is, in order to cause the residue to react in the low sugar concentration solution as described above, the residue need to have a uniform decomposition ratio. Therefore, in the present invention, in obtaining a high sugar concentration solution, the residue is separated in advance.

(Operation of First Embodiment)

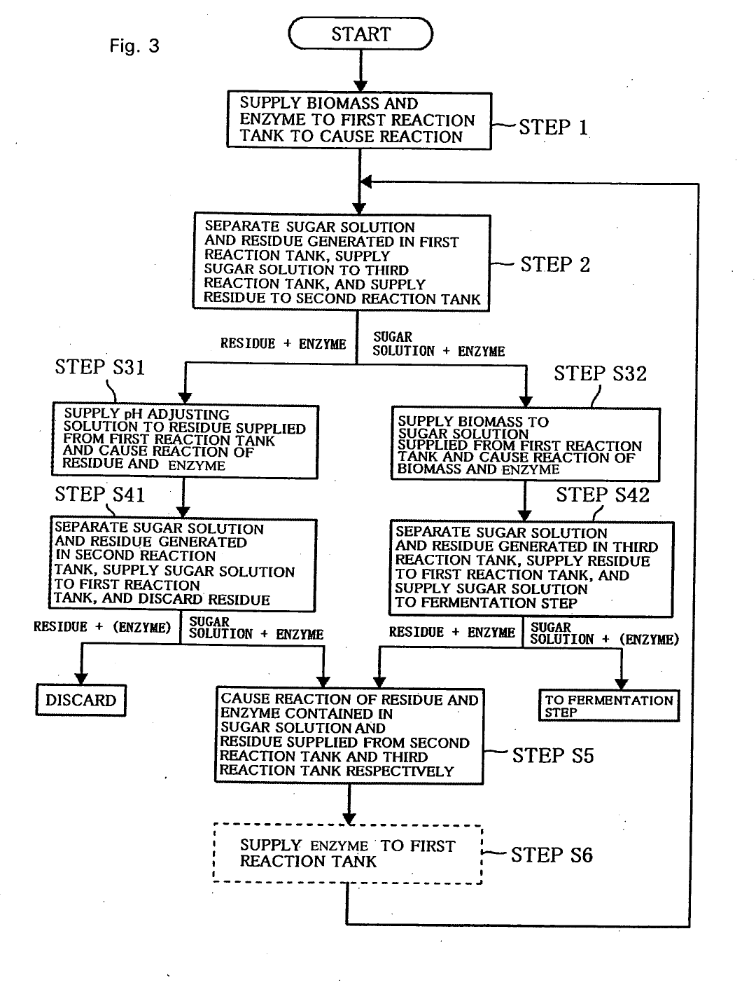

Based on the flow of the whole reaction shown in FIG. 3, it will be hereinafter described how biomass is decomposed, with a higher sugar concentration in the sugar solution that is taken to the outside, with a less amount of the residue discarded to the outside, and further, with a less amount of the enzyme discharged to the outside together with the sugar solution and the residue as described above, in the sugar solution producing method of the present invention where the biomass is decomposed based on the findings obtained by the above experiment results, compared with those of the device shown in FIG. 26.

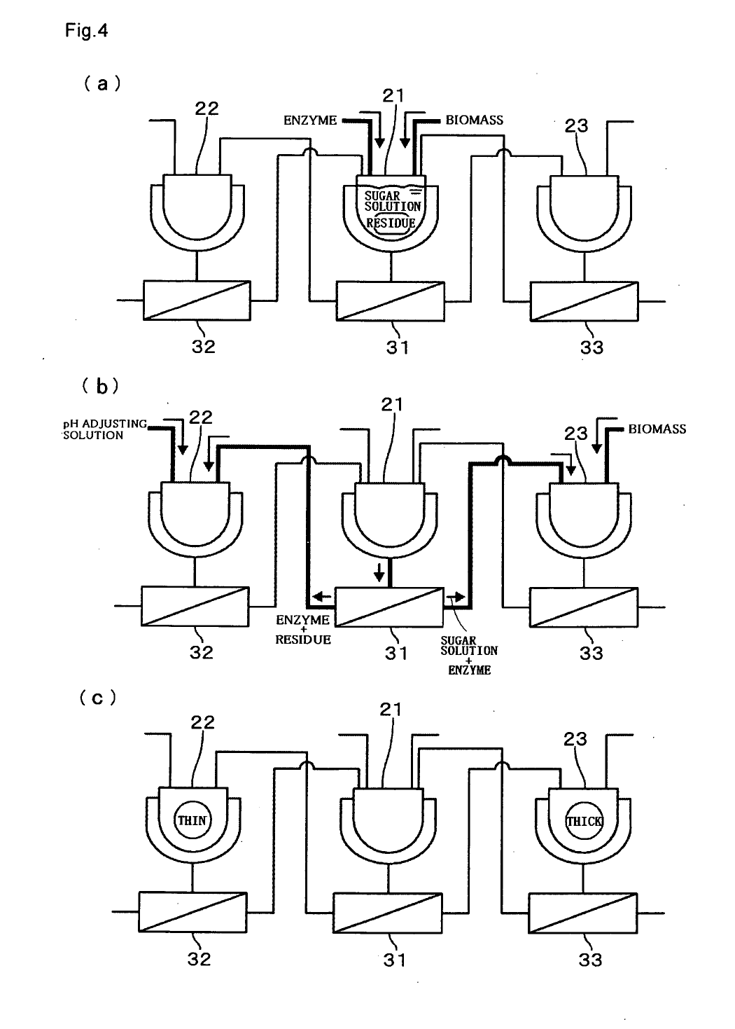

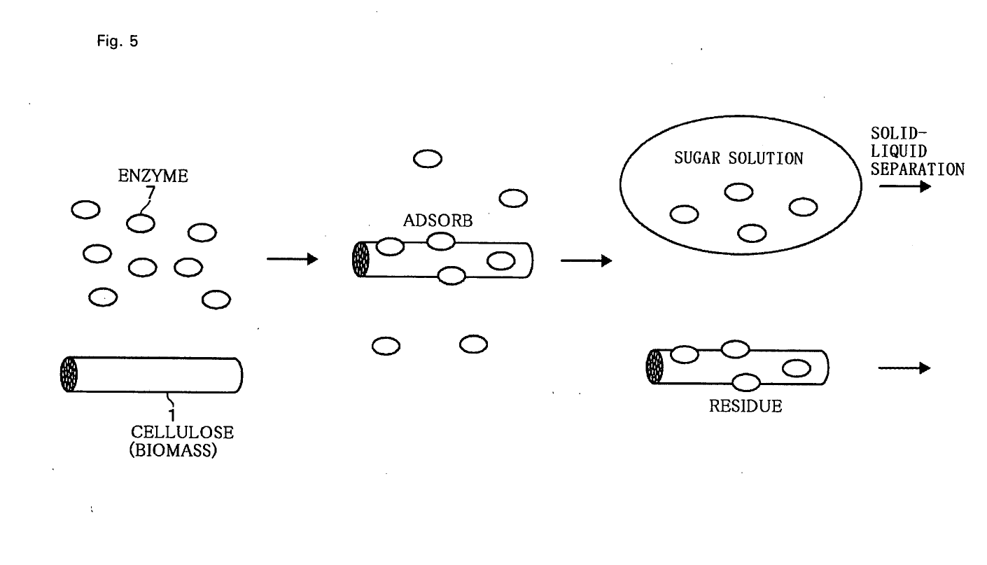

First, as shown in FIG. 4(a), as a first reaction step, enzyme (enzyme solution) and biomass having undergone the pretreatment are supplied into the first reaction tank 21 (Step S1). In a reaction solution of the first reaction tank 21, the enzyme 7 is adsorbed to the biomass (cellulose 1 and hemicellulose 2), so that a decomposition reaction progresses as shown in FIG. 5. The enzyme in the solution is not entirely absorbed by the biomass, but it partly exists in the solution. When an amount of the added enzyme is increased, an amount of the enzyme adsorbed to the biomass increases to enable an increase in a reaction rate, but at the same time, an enzyme concentration in the solution also increases. As a sugar concentration becomes higher in accordance with the progress of the reaction, an influence of sugar inhibition and a ratio of a portion not easily decomposed in a residue increase, so that the reaction rate gradually lowers. Next, as shown in FIG. 4(b), as a first separation step, a sugar solution and the residue which are generated in the first reaction tank 21 are separated in the first separator 31, and the sugar solution is supplied to the third reaction tank 23, and the residue is supplied to the second reaction tank 22 (Step S2). As shown in FIG. 5, the sugar solution and the residue separated in the first separator 31 both contain the enzyme as previously described.

Further, as shown in FIG. 4(b), as a second reaction step, a pH adjusting solution is supplied to the second reaction tank 22 to which the residue is supplied from the first reaction tank 21 (Step S31), and as a third reaction step, biomass is supplied to the third reaction tank 23 to which the sugar solution is supplied from the first reaction tank 21 (Step S32). As a result, as shown in FIG. 4(c), in the second reaction tank 22, a dilute solution whose sugar concentration is lower than that in the first reaction tank 21 is prepared, and accordingly the sugar inhibition decreases, so that the residue is quickly decomposed by the enzyme adsorbed to the residue. Therefore, in the second reaction tank 22, most of the cellulose 1 and the hemicellulose 2 in the residue supplied from the first reaction tank 21 is decomposed, so that the enzyme 7 adsorbed to the cellulose 1 and the hemicellulose 2 diffuses in the reaction solution and lignin 3 and so on contained in the residue slightly remain as a residue. At this time, the sugar concentration of a sugar solution obtained in the second reaction tank 22 is lower than the sugar concentration of the sugar solution obtained in the first reaction tank 21.

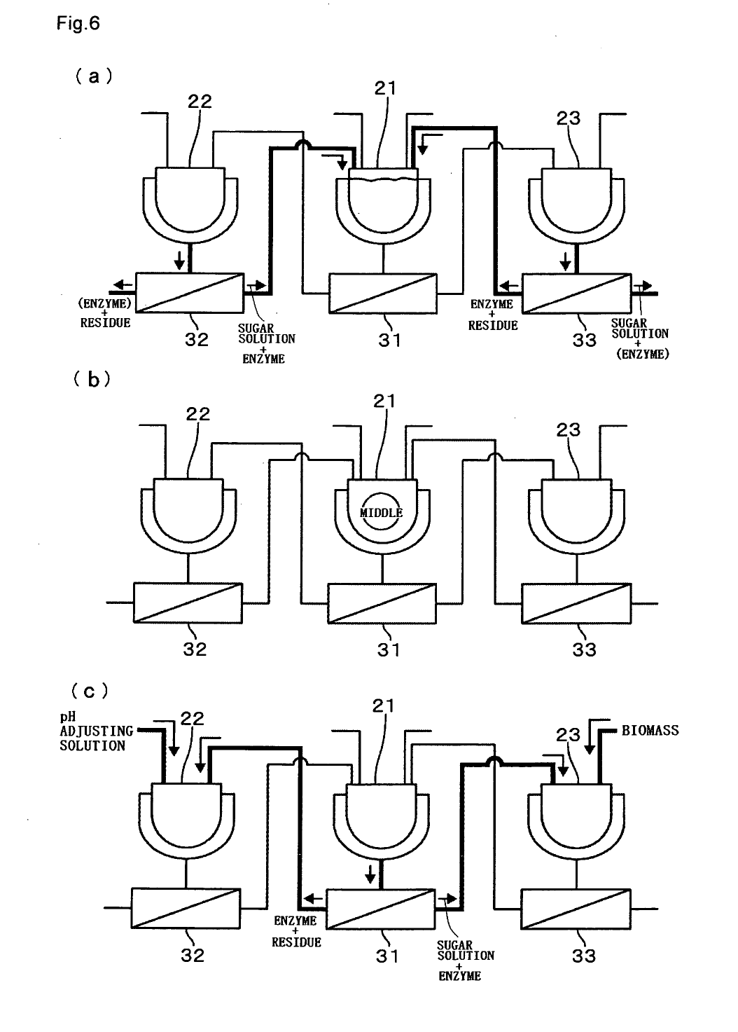

Meanwhile, in the third reaction tank 23, the enzyme contained in the sugar solution generated in the first reaction tank 21 is adsorbed to the added biomass, so that an enzyme concentration in a reaction solution quickly decreases. Then, in this third reaction tank 23, the sugar inhibition strongly works due to the sugar solution supplied from the first reaction tank 21, but an easily reacting portion in the newly added biomass is quickly decomposed by the enzyme in the sugar solution as previously described, so that a high-concentration sugar solution is generated. Subsequently, as shown in FIG. 6(a), as a second separation step, a sugar solution and a residue which are generated in the second reaction tank 22 are separated in the second separator 32, the sugar solution is supplied to the first reaction tank 21, and the residue is discarded to the outside of the system (Step S41). At this time, the enzyme adsorbed to the residue is discarded together with this residue, but since an amount of the residue has been greatly reduced by the reaction in the low sugar concentration solution as described above, most of the enzyme is supplied together with the sugar solution to the first reaction tank 21, so that an amount of the discarded enzyme is greatly reduced.

Meanwhile, in the third reaction tank 23, as a third separation step, an obtained high-concentration sugar solution and a residue are separated in the third separator 33, the high-concentration sugar solution is discharged to the outside of the system and the residue is supplied to the first reaction tank 21 (Step S42). The high-concentration sugar solution is discharged to the outside of the system together with the enzyme contained in the high-concentration sugar solution, but since most (about 60% from the aforesaid result in FIG. 20) of the enzyme in the third reaction tank 23 is absorbed by the unreacted biomass added to the third reaction tank 23, an amount of the discharged enzyme is small. This high-concentration sugar solution is sent to a not-shown fermentation tank, for instance, where it is fermented into alcohol, for example, ethanol or the like, and thereafter is condensed by distillation or the like.

Subsequently, in the first reaction tank 21, a reaction solution whose sugar concentration is lower than that of the high-concentration sugar solution obtained in the third reaction tank 23 is prepared from the sugar solution supplied from the second reaction tank 22 and the residue supplied from the third reaction tank 23 as shown in FIG. 6(b), so that the residue that cannot be decomposed due to the sugar inhibition in the third reaction tank 23 is given a less influence of the sugar inhibition and thus its decomposition progresses (Step S5). That is, this reaction step corresponds to the aforesaid reaction step at Step S1.

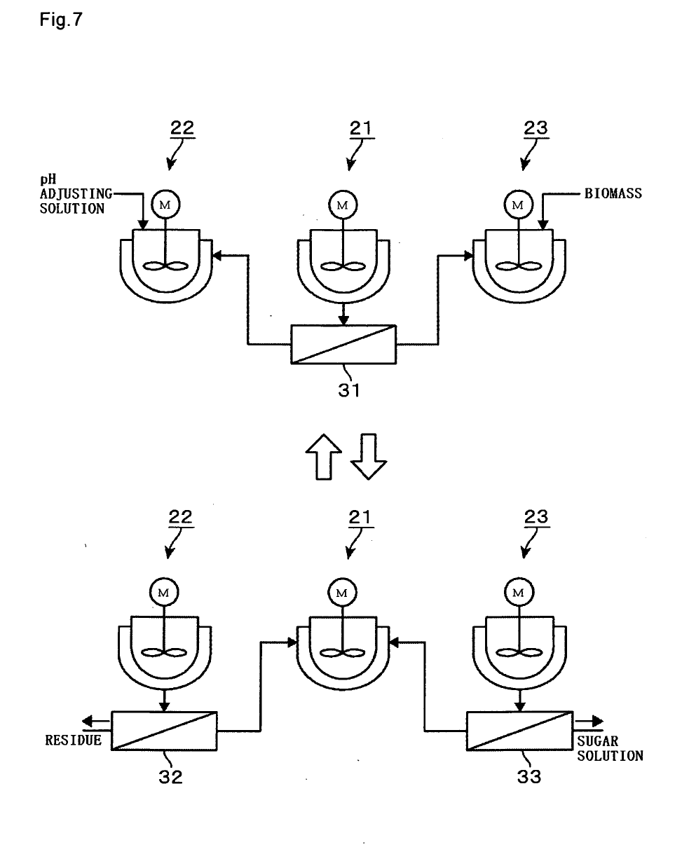

Thereafter, a sugar solution and a residue which are obtained in the first reaction tank 21 are supplied to the third reaction tank 23 and the second reaction tank 22 respectively, and the above-described Step S2 to Step S5 are repeated. Specifically, as shown in FIG. 7, the step of supplying the biomass and the pH adjusting solution to the sugar solution and the residue, which are obtained in the first reaction tank 21, in the third reaction tank 23 and the second reaction tank 22 respectively and the step of returning again the sugar solution and the residue obtained in the second reaction tank 22 and the third reaction tank 23 respectively to the first reaction tank 21 are alternately repeated, whereby high-concentration sugar solutions are continuously obtained. Therefore, in this device, the biomass and the pH adjusting solution are, so to speak, intermittently supplied to the third reaction tank 23 and the second reaction tank 22.

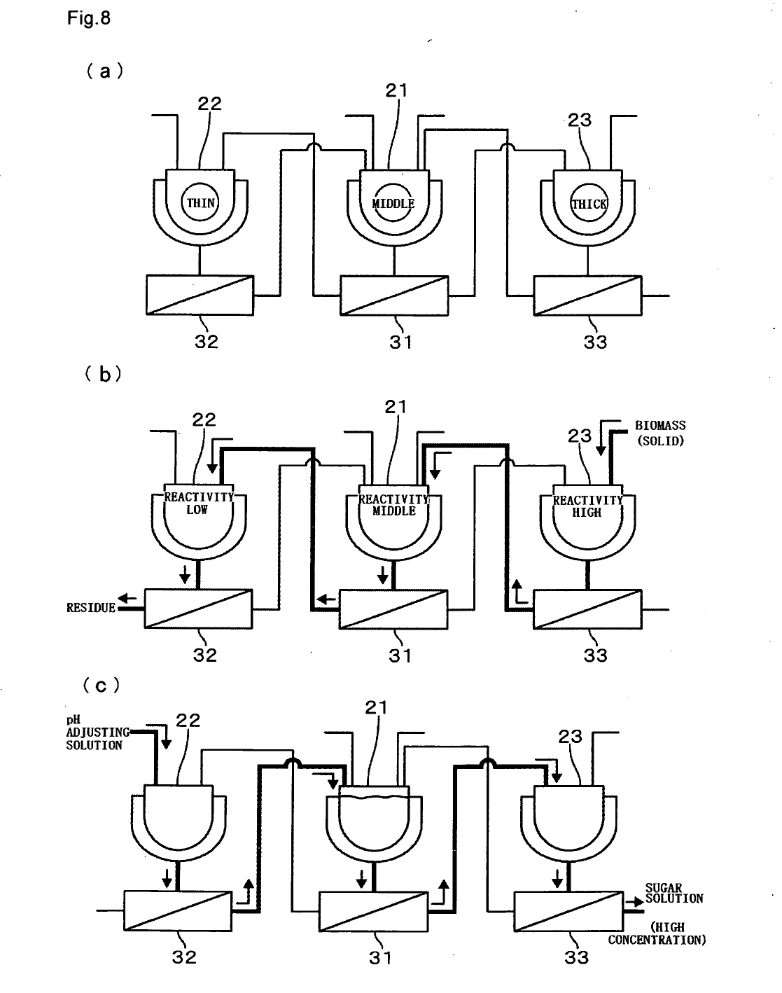

Then, in the reaction tanks 10, reaction solutions different in sugar concentration are prepared respectively as previously described, that is, as shown in FIG. 8(a), in the three reaction tanks 10, the sugar concentration becomes higher in order from the left second reaction tank 22 toward the right third reaction tank 23. Therefore, a degree of the sugar inhibition becomes higher in order from the left second reaction tank 22 toward the right third reaction tank 23, and accordingly, as shown in FIG. 8(b), the biomass added to the right third reaction tank 23 decomposes in order from the place where it has higher reactivity (place where the influence of the sugar inhibition is smaller) toward the left second reaction tank 22. Further, the pH adjusting solution supplied to the left second reaction tank 22 gradually becomes higher in sugar concentration as it goes toward the right third reaction tank 23, and is taken out of the system as the high-concentration sugar solution in the third separator 33.

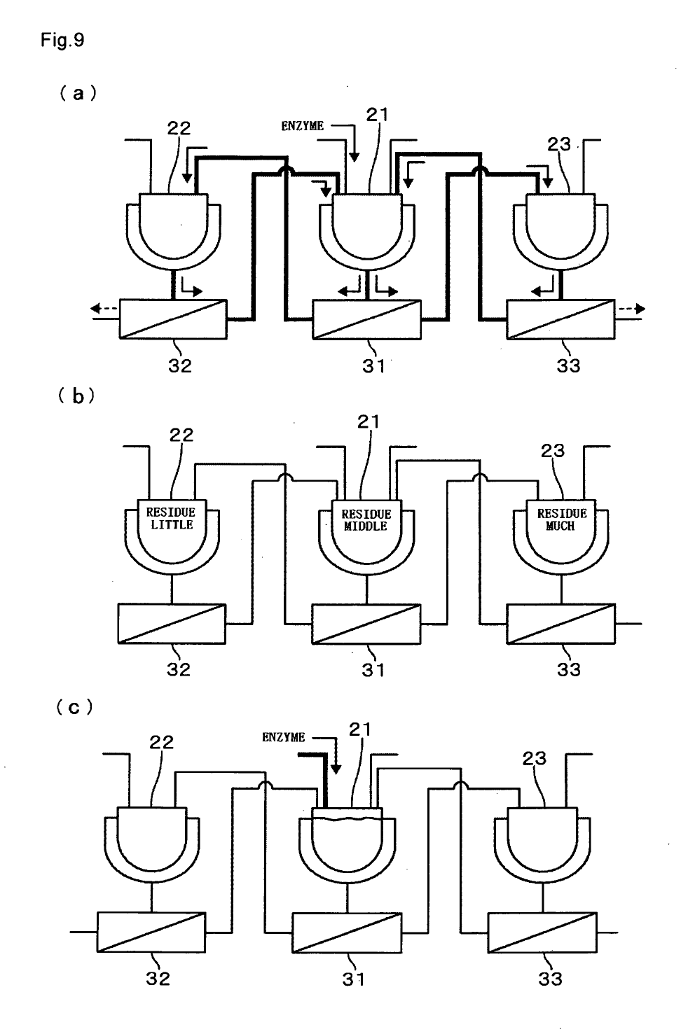

Part of the enzyme supplied to the first reaction tank 21 is adsorbed to the residue to be discharged from the second reaction tank 22 to the outside of the system and some other part thereof is discharged to the outside of the system from the third reaction tank 23 together with the high-concentration sugar solution, but by adjusting operation conditions in the respective reaction tanks 10 so that an amount of the generated residue becomes small in the second reaction tank 22 and an amount of the enzyme adsorbed to the residue and returned to the first reaction tank 21 becomes large in the third reaction tank 23, the enzyme, so to speak, circulates among the three reaction tanks 10 as shown in FIG. 9(a). That is, it can be said that amounts of the residue in the three reaction tanks 10 are adjusted as shown in FIG. 9(b) in order to utilize the nature that the enzyme is adsorbed to the biomass (residue) to return to the reaction solution again due to the decomposition of the biomass. Concretely, an amount of the residue is adjusted so that it becomes larger in order from the left second reaction tank 22 toward the right third reaction tank 23. Therefore, it can be said that the second reaction tank 22 has a function of collecting the enzyme from the residue and the third reaction tank 23 has a function of collecting the enzyme from the reaction solution.

At this time, since the enzyme is also discharged to the outside of the system, though only slightly, and since the enzyme sometimes becomes deactivated by the decomposition reaction of the biomass, the enzyme is additionally supplied to the first reaction tank 21 in these cases as shown in FIG. 9(c) (Step S6).

Here, when the above steps are automated, a not-shown control part is provided in this saccharification device, and the control part outputs a control signal to the saccharification device so that the aforesaid allowance/stop of the supply by the not-shown valve and the agitating and heating of the reaction solutions in the reaction tanks 21 to 23 are performed in order for the above-described steps to be performed.

According to the above-described embodiment, in obtaining the sugar solution through the reaction of the enzyme and the biomass containing cellulose, the sugar solution in which the enzyme is dispersed and the residue containing the unreacted biomass adsorbing the enzyme are generated through the reaction of the biomass and the enzyme in the first reaction tank 21, next, these sugar solution and residue are separated, and the pH adjusting solution is supplied to the residue in the second reaction tank 22, whereby the dilute solution whose sugar concentration is lower than that of the aforesaid sugar solution is prepared, and the residue and the enzyme adsorbed to the residue are made to react in this dilute solution, whereby the sugar solution is obtained. Therefore, it is possible to efficiently use and collect the enzyme and also to reduce an amount of the discarded residue (obtain a high saccharification ratio), which makes it possible to obtain the sugar solution at low cost. Further, since the recovery and reuse of the enzyme are achieved, it is possible to, for example, increase an amount of the supplied enzyme, in which case the sugar solution can be obtained in a short time.

Further, in the third reaction tank 23, by further adding the biomass to the sugar solution which has been separated from the residue, it is possible for the enzyme dispersed in the sugar solution to react with the easily decomposed portion in the newly added biomass, which enables efficient use of the enzyme and makes it possible to quickly increase the sugar concentration in the sugar solution. Furthermore, it is possible to collect the enzyme dispersed in the sugar solution by the residue of the newly added biomass adsorbing it. Achieving the production of the sugar solution with a high sugar concentration and a reduction in an amount of the discharged enzyme allow each of the reaction tanks 10 to be small, enabling a reduction in facility expense, can reduce energy required for the later distillation, and further can reduce the expense for the enzyme, which makes it possible to obtain the sugar solution and ethanol at low cost. At this time, since the residue generated in the first reaction tank 21 is separated in advance before the biomass is added to the third reaction tank 23, the decomposition ratio of the residue generated in the third reaction tank 23 can be uniform, and accordingly, the residue can be efficiently decomposed in the first reaction tank 21 and the second reaction tank 22 to which the residue is sequentially supplied thereafter.

Then, since the sugar solution and the residue generated in the second reaction tank 22 and the third reaction tank 23 respectively are returned again to the first reaction tank 21, it is possible to continuously obtain the high-concentration sugar solutions by making an effective use of these sugar solution and residue and to reduce amounts of the discarded residue and enzyme. Further, the use of the three reaction tanks 10 for the above-described processes enables the efficient performance of the processes. Further, in circulating (reusing) the enzyme in the three reaction tanks 10, no consumables such as, for example, a membrane, is not used, it is possible to obtain the high-concentration sugar solution at low cost.

Further, in the second reaction tank 22, the enzyme is discharged to the outside of the system together with the residue, but since this residue is mainly lignin, by making a content of the lignin in the supplied biomass small in advance as previously described, it is possible to reduce an amount of the enzyme discharged together with the residue.

In additionally supplying the enzyme at the aforesaid Step S6, the enzyme is supplied to the first reaction tank 21 but it may be supplied to the second reaction tank 22.

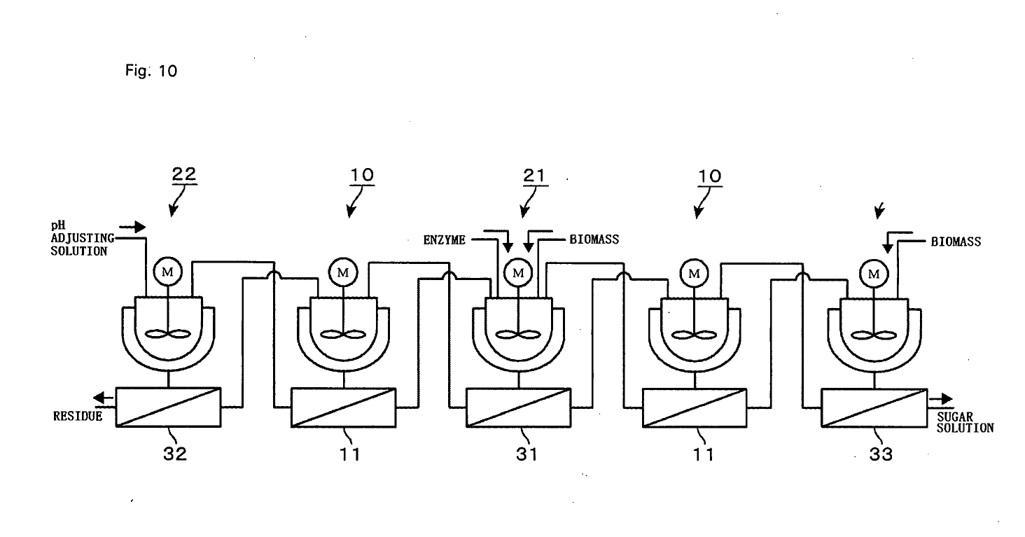

Further, in the above-described example, the three reaction tanks 10 are provided, but the number thereof may be three or more, for example, five as shown in FIG. 10, for instance. In this case, the sugar concentration of the pH adjusting solution supplied to the second reaction tank 22 gradually increases as it goes toward the right reaction tank 23. Accordingly, since the biomass supplied to the third reaction tank 23 in which the sugar concentration is extremely high is strongly influenced by the sugar inhibition, a portion with extremely high reactivity in the biomass reacts, and most of the enzyme in the third reaction tank 23 is recovered together with the residue from the third reaction tank 23. Further, since the sugar concentration decreases from the right third reaction tank 23 toward the left second reaction tank 22, the biomass with extremely low reactivity is supplied as the residue to the second reaction tank 22 and is decomposed in the second reaction tank 22. Accordingly, an amount of the residue discharged to the outside of the system from the second reaction tank 22 becomes a trace amount, which in turn reduces an amount of the enzyme discharged together with the residue. Therefore, as the number of the reaction tanks 10 is larger, the sugar solution with a higher concentration than that in the above-described example is obtained, and further, an amount of the residue and an amount of the enzyme that are discarded become further smaller.

Further, the separators 11 are provided for the respective reaction tanks 10, but the same separator 11 may be commonly used for the reaction tanks. Another alternative structure may be, for example, to settle down or filtrate the residue in each of the reaction tanks 10, and for example, suck a supernatant liquid to take out a deposit and a filtrated substance (residue) or leave them in the reaction tank. In this case, the reaction tanks 10 each also serve as a separating means.

### Second Embodiment

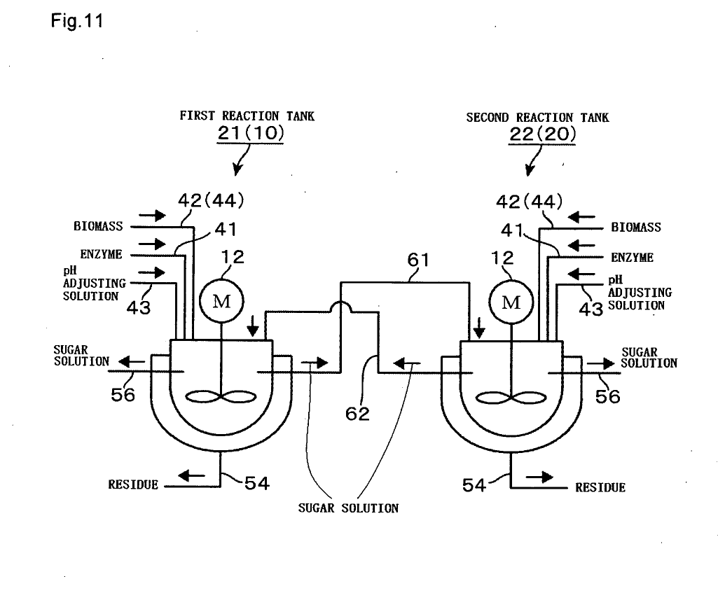

Alternatively, the number of the reaction tanks 10 may be two. Such a sugar solution producing device will be described with reference to FIG. 11, taking as an example a case where a residue is settled down in the above-described manner, and two first reaction tank 21 and second reaction tank 22 also serve as the first separator 31 and the second separator 32 respectively. The same portions as those of the above-described embodiment will be denoted by the same reference numerals and description thereof will be omitted.

Regarding the two reaction tanks 21, 22, for convenience sake, the left one is called a first reaction tank 21 and the right one is called a second reaction tank 22, and an enzyme supply path 41, a biomass supply path 42 (biomass adding path 44), a pH adjusting solution supply part 43, a residue discharge path 54, and a sugar solution recovery path 56 are connected to each of the reaction tanks 21, 22. In the description below, “first” is appended to the supply paths and the recovery path (the enzyme supply path 41, the biomass supply path 42 (biomass adding path 44), the pH adjusting solution supply path 43, the residue discharge path 54, and the sugar solution recovery path 56 connected to the first reaction tank 21 and “second” is appended to those connected to the second reaction tank 22. A first supply path (first transfer part) 61 and a second supply path 62 for sucking sugar solutions being supernatant liquids in the reaction tanks 21, 22 to supply them to the other reaction tanks 22, 21 are connected to the reaction tanks 21, 22.

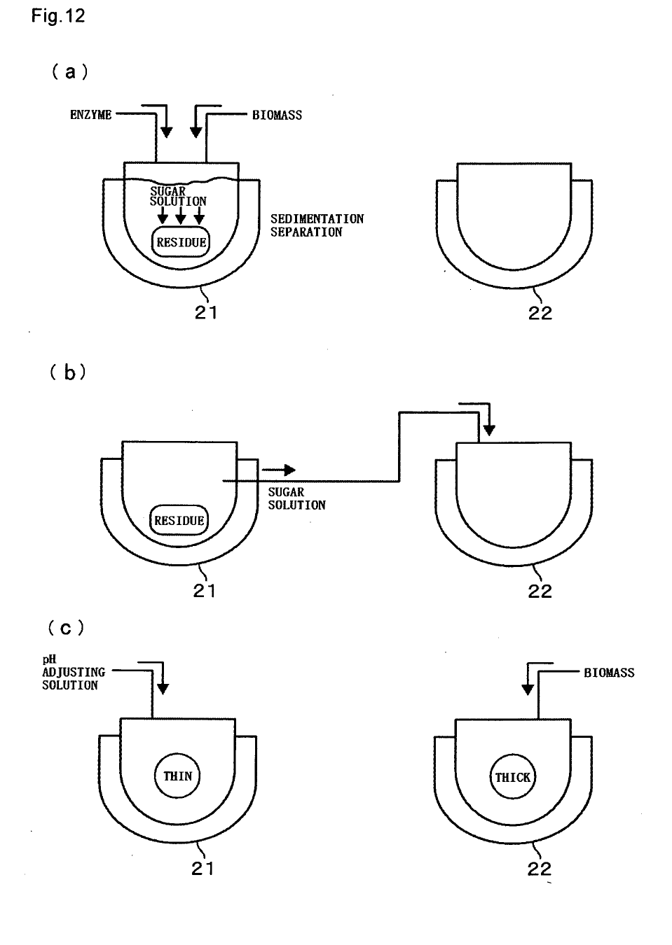

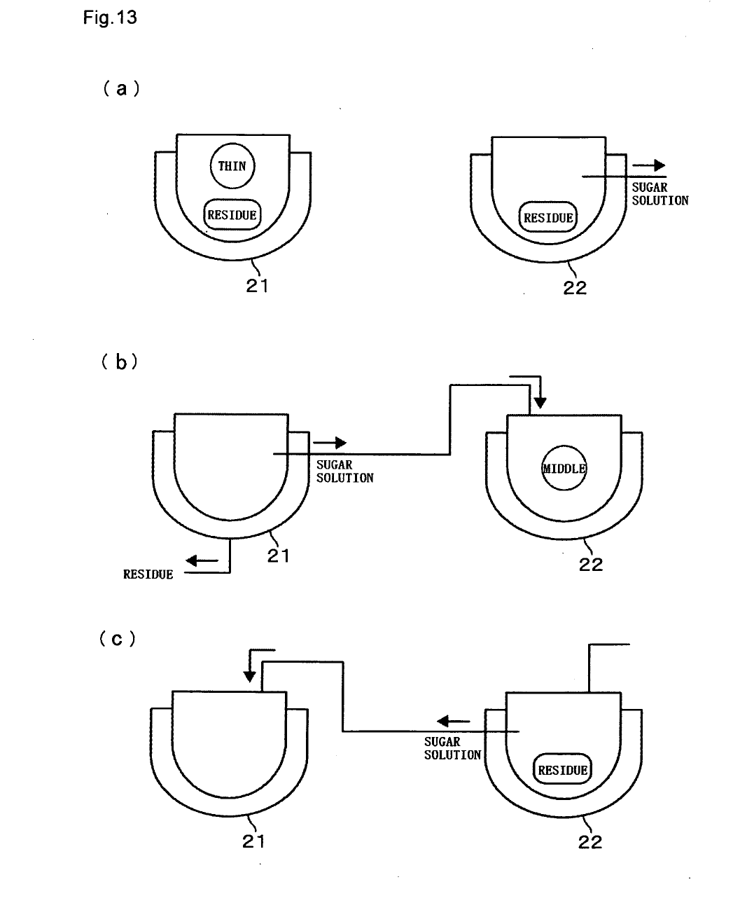

In this sugar solution producing device, first, when enzyme, a pH adjusting solution, and biomass are supplied to the first reaction tank 21, a sugar solution and a residue are generated, as shown in FIG. 12(a) (Step S1). In this first reaction tank 21, the sugar solution and the residue are separated by, for example, sedimentation, and as shown in FIG. 12(b), the sugar solution is supplied to the second reaction tank 22 (Step S2). Then, as shown in FIG. 12(c), a pH adjusting solution is supplied to the first reaction tank 21 where the residue is left (Step S31), and biomass is supplied to the second reaction tank 22 (Step S32). Next, sugar solutions and residues generated in the reaction tanks 21, 22 respectively are separated by sedimentation, and as shown in FIG. 13(a), the high-concentration sugar solution generated in the second reaction tank 22 is taken to the outside of the system (Step S42). Then, as shown in FIG. 13(b), the sugar solution generated in the first reaction tank 21 is supplied to the second reaction tank 22 and is mixed with the residue remaining in the second reaction tank 22, and at the same time, the residue remaining in the first reaction tank 21 is discharged to the outside of the system (Step S41). In the second reaction tank 22, since the residue and the sugar solution react with each other (Step S5), the above-described processes at Step S2 (FIG. 13(c)) to Step S5 are applied to these sugar solution and residue. In this example, the high-concentration sugar solution and a dilute solution are alternately prepared in the first reaction tank 21 and the second reaction tank 22. Further, when there occurs a shortage of the enzyme, the enzyme is additionally supplied to either of the reaction tanks 21, 22.

In this embodiment, the same operations and effects as those of the above-described embodiment can also be obtained.

In the above-described case, the residue is left and the sugar solution is moved to/from the reaction tanks 21, 22, but another possible structure may be, for example, to move the residue, which has been separated by sedimentation, by sucking the residue from under. In this case, the same operations and effects are also obtained.

Further, the biomass used as a raw material of the sugar solution in each of the above-described examples may be a processed product of a plant raw material, such as used paper, pulp, or cotton fiber, containing the cellulose 1, instead of a woody raw material and a herbaceous raw material, or may be comprised of a plurality of kinds of these raw materials containing the cellulose 1. The enzyme is preferably a cellulose decomposing enzyme or a combination of a cellulose decomposing enzyme and a hemicellulose decomposing enzyme. Further, in the production of the sugar solution in the above-described manner, the present invention includes a method where the sugar solution and the residue are generated through the reaction of the biomass and the enzyme outside the system in advance at an initial stage, and at the time of continuous operation, the sugar solution and the residue are supplied to the different reaction tanks 10 respectively and the above-described processes are performed.

### Example

Next, regarding the results of the various experiments already described in detail, conditions, brief results, and so on of the experiments will be described below.

FIG. 14: Experiment for Evaluating Correlation between Reaction Time and Sugar Concentration

(experiment condition)

substrate (biomass): filter paper 10 g

enzyme: 5 ml

buffer solution: 95 ml

(experiment result)

A decomposition rate (an increasing rate of the sugar concentration) decreased with time.

FIG. 15: Experiment for Evaluating Correlation Between Amount of Raw Material Charged to Reaction Tank and Saccharification Ratio (Sugar Yield)

(experiment condition)

substrate (biomass): filter paper

charge amount of substrate: 10 g, 25 g, 30 g, 35 g, 40 g

enzyme: 5 ml

buffer solution: 95 ml

reaction temperature: 50°

(experiment result)

As the charge amount of the raw material increased, the saccharification ratio decreased.

FIG. 16: Experiment for Evaluating Time-Dependent Change of Enzyme Concentration when Biomass is Supplied to Enzyme Solution

(experiment condition)

substrate (biomass): filter paper 10 g

enzyme: 5 ml

buffer solution: 95 ml

(experiment result)

When the biomass was supplied to the enzyme solution, the concentration of the enzyme (protein) in a solution decreased.

FIG. 17: Experiment for Evaluating Time-Dependent Change of Enzyme Concentration when Biomass is Supplied to Enzyme Solution.

(experiment condition)

substrate (biomass): filter paper 10 g

enzyme: 5 ml

pH adjusting solution: 95 ml

(experiment result)

When the biomass was supplied to the enzyme solution, the enzyme was adsorbed to the biomass and accordingly an amount of the enzyme in the solution decreased, but in a short time thereafter, the enzyme adsorbed to the biomass decomposed to return again into the solution.

FIG. 18: Experiment for Confirming Change in Sugar Concentration when Residue is Supplied into Buffer Solution (Low Sugar Concentration Solution)

(experiment condition)

substrate (biomass): a residue generated under the following residue generation condition and centrifuged 16 g (wet state)

reaction solution: the above residue+buffer solution 50 ml
(residue generation condition)
substrate (biomass): filter paper 10 g
enzyme: 5 ml
buffer solution: 95 ml
saccharification time: 168 h

(experiment result)

The decomposition reaction of even the residue whose decomposition reaction did not easily progress progressed quickly when it was separated from a sugar solution whose sugar concentration became high and supplied into a low sugar concentration solution.

FIG. 19: Experiment for Confirming Change in Sugar Concentration when Residue is Supplied into Buffer Solution (Low Sugar Concentration Solution)

(experiment condition)

substrate (biomass): a residue obtained through centrifugal separation of the reaction solution 6 days later from the start of the reaction in the experiment in FIG. 14 10 g

reaction solution: the above residue+buffer solution 100 ml

(experiment result)

The decomposition reaction of even the residue whose decomposition reaction did not easily progress similarly progressed quickly in the low sugar concentration solution.

FIG. 20: Experiment for Confirming Change in Enzyme Concentration in Sugar Solution when Biomass is Supplied to Sugar Solution

(experiment condition)

substrate (biomass): filter paper 10 g

reaction solution: a sugar solution obtained through centrifugal separation of the reaction solution six days later from the start of the experiment in the experiment under the same condition as that in FIG. 14 100 ml

(experiment result)

When the biomass was supplied to the sugar solution whose decomposition reaction was delayed due to the occurrence of the sugar inhibition, the enzyme (protein) concentration in the sugar solution decreased.

FIG. 21: Experiment for Evaluation Change in Sugar Concentration when Biomass is Supplied to Sugar Solution

(experiment condition)

substrate (biomass): filter paper 10 g

reaction solution: a sugar solution obtained through centrifugal separation of the reaction solution one day later from the start of the experiment in the experiment under the same condition as that in FIG. 14 100 ml

(experiment result)

Even in a sugar solution whose sugar concentration became high to such a degree as to make the decomposition reaction difficult to progress due to the sugar inhibition, the biomass was quickly decomposed and the sugar concentration increased when the biomass was newly supplied.

FIG. 22: Experiment for Evaluating Change in Sugar Concentration when Biomass is Supplied to Sugar Solution

(experiment condition)

substrate (biomass): filter paper 10 g

reaction solution: a sugar solution obtained through centrifugal separation of the reaction solution six days later from the start of the experiment in the experiment under the same condition as that in FIG. 14 100 ml

(experiment result)

As in the above experiment, even in a sugar solution whose sugar concentration became high, the biomass was quickly decomposed and the sugar concentration increased when the biomass was newly supplied.

FIG. 23: Experiment for Evaluating Change in Sugar Concentration when Biomass is Added in the Course of Reaction

(experiment condition)

substrate (biomass): filter paper

substrate concentration: 10 weight/vol %

enzyme: 5 vol %

pH adjusting solution: 50 mM acetic acid buffer solution pH5

reaction temperature 50° C.

(experiment result)

When a 10 weight/vol % filter paper was newly added 50 h later from the start of the experiment, the sugar concentration whose increasing rate became low before the addition quickly increased.

FIG. 24: Experiment for Evaluating Change in Saccharification Ratio when Biomass is Added in the Course of Reaction

(experiment condition)

substrate (biomass): filter paper

substrate concentration: 10 weight/vol %

enzyme: 5 vol %

pH adjusting solution: 50 mM acetic acid buffer solution pH5

reaction temperature: 50° C.

(experiment result)

When a 10 weight/vol % filter paper was newly added 50 h later from the start of the experiment, the saccharification ratio of the biomass after the addition was lower than that before the addition.

### EXPLANATION OF REFERENCES

1 cellulose
2 hemicellulose
7 enzyme
21 first reaction tank
22 second reaction tank
23 third reaction tank
31 to 33 separator
41 enzyme supply path
42 biomass supply path
43 pH adjusting solution supply path
44 biomass adding path

1. A method of producing a sugar solution to obtain a sugar solution through a reaction of cellulose-based biomass being an aggregate of plants or an aggregate of processed products of plant including cellulose with a saccharifying enzyme having cellulose saccharifying ability, the method comprising:
a first reaction step of mixing a saccharifying enzyme and cellulose-based biomass in an aqueous solution to cause a saccharification of the biomass by the saccharifying enzyme;
a first separation step of causing solid-liquid separation of a reaction solution obtained in the first reaction step to obtain a sugar solution and a residue; and
a second reaction step of preparing an aqueous solution by adding additive water to the residue obtained in the first separation step, and in the aqueous solution, causing a saccharification of the residue by the saccharifying enzyme adsorbed to the residue; and
a third reaction step of adding cellulose-based biomass not having undergone the saccharification to the sugar solution obtained in the first separation step and causing a saccharification of the newly added biomass by the saccharifying enzyme present in the sugar solution.

2. The method of producing the sugar solution according to claim 1, wherein different reaction tanks are used for the first reaction step, the second reaction step, and the third reaction step.

3. The method of producing the sugar solution according to claim 1, comprising:
a second separation step of causing solid-liquid separation of a reaction solution obtained in the second reaction step to obtain a sugar solution and a residue;
a third separation step of causing solid-liquid separation of a reaction solution obtained in the third reaction step to obtain a high-concentration sugar solution and a residue; and
a reaction step of mixing the sugar solution obtained in the second separation step and the residue obtained in the third separation step to cause a saccharification,
wherein the reaction step corresponds to the first reaction step, and the biomass used in the first reaction step is the residue obtained in the third separation step.

4. The method of producing the sugar solution according to claim 3, further comprising
a step of additionally supplying a saccharifying enzyme to at least one of the aqueous solution in the second reaction step and the sugar solution separated in the second separation step.

5. The method of producing the sugar solution according to claim 1, wherein a ratio of lignin contained in the biomass is 10% or less.

6. A saccharification device for carrying out the method of producing the sugar solution according to claim 1, the device comprising:
two reaction tanks in each of which a saccharifying enzyme and a cellulose-based biomass are mixed in an aqueous solution and a saccharification of the biomass is caused by the saccharifying enzyme;
a biomass supply part to supply the biomass into the reaction tanks;
a first enzyme supply part to supply the saccharifying enzyme into the reaction tanks;
an additive water supply part to supply additive water into one reaction tank out of the two reaction tanks;
a first separating part to perform solid-liquid separation of a reaction solution generated by the reaction between the biomass and the saccharifying enzyme in the one reaction tank to obtain a sugar solution and a residue;
a first transfer part to transfer the sugar solution separated by the first separating part to the other reaction tank out of the two reaction tanks; and
a control part that outputs a control signal so that the first reaction step is performed in the one reaction tank, the sugar solution obtained from the first separating part is next supplied to the other reaction tank, thereafter the additive water and cellulose-based biomass not having undergone the saccharification are supplied into the reaction tank storing the residue and the reaction tank storing the sugar solution respectively, and the second reaction step and the third reaction step are performed in the two reaction tanks respectively.

7. The saccharification device according to claim 6,
wherein the first enzyme supply part supplies the saccharifying enzyme into the one reaction tank instead of the two reaction tanks,
wherein the additive water supply part supplies the additive water into the other reaction tank instead of the one reaction tank,
wherein the first transfer part transfers the residue instead of the sugar solution to the other reaction tank, and
wherein the control part outputs the control signal so that after the first reaction step is performed, the other reaction tank is supplied with the residue obtained by the first separating part instead the sugar solution obtained by the first separating part.

8. The saccharification device according to claim 7, wherein the first separating part separates the sugar solution and the residue by sedimentation in the one reaction tank.

9. A saccharification device for carrying out the method of producing the sugar solution according to claim 1, the device comprising:
a first reaction tank, a second reaction tank, and a third reaction tank in each of which a saccharifying enzyme and the cellulose-based biomass are mixed in an aqueous solution and a saccharification of the biomass is caused by the saccharifying enzyme;
biomass supply parts to supply the biomass to the first reaction tank and the third reaction tank respectively;
an enzyme supply part to supply the saccharifying enzyme into the first reaction tank;
an additive water supply part to supply additive water into the second reaction tank;
a first separating part to perform solid-liquid separation of a reaction solution obtained by the reaction between the saccharifying enzyme and the biomass in the first reaction tank to obtain a sugar solution and a residue;
a first sugar solution supply part and a first residue supply part to supply the sugar solution and the residue separated by the first separating part to the third reaction tank and the second reaction tank respectively; and
a control part that outputs a control signal so that the first reaction step is performed in the first reaction tank, next, the residue and the sugar solution are separated by the first separating part from the reaction solution obtained in the first reaction tank, the residue and the sugar solution are supplied to the second reaction tank and the third reaction tank respectively, and thereafter, the additive water and cellulose-based biomass not having undergone the saccharification are supplied to the second reaction tank and the third reaction tank respectively, and the second reaction step and the third reaction step are performed in the reaction tanks respectively.

10. The saccharification device according to claim 9, comprising:
a second separating part to perform solid-liquid separation of a reaction solution generated in the second reaction tank to obtain a sugar solution and a residue;
a third separating part to perform solid-liquid separation of a reaction solution generated in the third reaction tank to obtain a high-concentration sugar solution and a residue;
a second sugar solution supply part to supply the first reaction tank with the sugar solution separated in the second separating part;
a third residue supply part to supply the first reaction tank with the residue separated in the third separating part;
a sugar solution collecting part to take out the high-concentration sugar solution separated in the third separating part, and
wherein there is performed a step of obtaining a sugar solution and a residue from a reaction solution obtained in the second reaction tank, by using the second separating part, obtaining a high-concentration sugar solution and a residue from a reaction solution obtained in the third reaction tank, by using the third separating part, and making the sugar solution separated in the second separating part react in the first reaction tank with the residue separated in the third separating part, and
wherein the step corresponds to the first reaction step, and the biomass used in the first reaction step is the residue separated in the second separating part.

11. The saccharification device according to claim 10, wherein the first separating part, the second separating part, and the third separating part separate the sugar solution and the residue by sedimentation in the first reaction tank, the second reaction tank, and the third reaction tank respectively.

12. The saccharification device according to claim 6, wherein a ratio of lignin contained in the biomass is 10% or less.

13. The saccharification device according to claim 9, wherein a ratio of lignin contained in the biomass is 10% or less.

14. A saccharification device for producing a sugar solution comprising:
two reaction tanks in each of which a saccharifying enzyme and a cellulose-based biomass are mixed in an aqueous solution and a saccharification of the biomass is caused by the saccharifying enzyme;
a biomass supply part to supply the biomass into the reaction tanks;
a first enzyme supply part to supply the saccharifying enzyme into the reaction tanks;
an additive water supply part to supply additive water into one reaction tank out of the two reaction tanks;
a first separating part to perform solid-liquid separation of a reaction solution generated by the reaction between the biomass and the saccharifying enzyme in the one reaction tank to obtain a sugar solution and a residue;
a first transfer part to transfer the sugar solution separated by the first separating part to the other reaction tank out of the two reaction tanks; and
a control part that outputs a control signal so that the first reaction step is performed in the one reaction tank, the sugar solution obtained from the first separating part is next supplied to the other reaction tank, thereafter the additive water and cellulose-based biomass not having undergone the saccharification are supplied into the reaction tank storing the residue and the reaction tank storing the sugar solution respectively, and the second reaction step and the third reaction step are performed in the two reaction tanks respectively.

15. A saccharification device for producing a sugar solution comprising:
a first reaction tank, a second reaction tank, and a third reaction tank in each of which a saccharifying enzyme and the cellulose-based biomass are mixed in an aqueous solution and a saccharification of the biomass is caused by the saccharifying enzyme;
biomass supply parts to supply the biomass to the first reaction tank and the third reaction tank respectively;
an enzyme supply part to supply the saccharifying enzyme into the first reaction tank;
an additive water supply part to supply additive water into the second reaction tank;
a first separating part to perform solid-liquid separation of a reaction solution obtained by the reaction between the saccharifying enzyme and the biomass in the first reaction tank to obtain a sugar solution and a residue;
a first sugar solution supply part and a first residue supply part to supply the sugar solution and the residue separated by the first separating part to the third reaction tank and the second reaction tank respectively; and
a control part that outputs a control signal so that the first reaction step is performed in the first reaction tank, next, the residue and the sugar solution are separated by the first separating part from the reaction solution obtained in the first reaction tank, the residue and the sugar solution are supplied to the second reaction tank and the third reaction tank respectively, and thereafter, the additive water and cellulose-based biomass not having undergone the saccharification are supplied to the second reaction tank and the third reaction tank respectively, and the second reaction step and the third reaction step are performed in the reaction tanks respectively.
